(19) United States
(12) Patent Application Publication
(10) Pub. No.: US 2005/0130131 A1
(43) Pub. Date: Jun. 16, 2005

(54) Method for isolation and replication of infectious human hepatitis-C virus

(75) Inventor: Syed Zaki Salahuddin, Ventura, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: California Institute of Molecular Medicine, Ventura, CA (US)

(21) Appl. No.: 10/920,040

(22) Filed: Aug. 16, 2004

Related U.S. Application Data
(60) Provisional application No. 60/495,078, filed on Aug. 14, 2003.

(51) Int. Cl. C12Q 1/70; C12Q 1/68; C07H 21/04; C12N 5/08; C12N 15/86

(52) U.S. Cl. 435/5; 435/6; 435/235.1; 435/456; 435/372; 536/23.72

(57) ABSTRACT

The present invention provides methods and compositions for replicating infectious Hepatitis C virus in vitro.

### CROSS-REFERENCES TO RELATED APPLICATIONS

This application claims the benefit of U.S. Provisional Patent Application No. 60/495,078, filed Aug. 14, 2003, the disclosure of which is hereby incorporated by reference in its entirety for all purposes.

### BACKGROUND OF THE INVENTION

The global public health impact of chronic Hepatitis C virus (“HCV”) infection and consequent liver disease continues to grow in numbers. It has been estimated that there are over 170 million carriers of HCV worldwide, with an increasing incidence of new infections (World Health Organization, Weekly Epidemiol Rec. 72:341-344 (1997)). In the United States, an estimated 2.7 million individuals are currently chronically infected with this virus (Salomon et al., JAMA 290:228-237 (2003)).

Although HCV has proven to be very difficult to grow in vitro, HCV-RNA has been detected in cell cultures of a variety of cell types for periods ranging from a few days to several months, albeit with no evidence of infectious virus (Iacovacci et al., Res Virol. 144:275-279 (1993); Morsica et al., Blood 94:1138-1139 (1999); Shimizu et al., PNAS USA 89:5477-5481 (1992); Sung et al., J. Virol. 77:2134-2146 (2003)). The recent creation of HCV-RNA replicons has contributed to a better understanding of some of the molecular events, particularly gene expression (Blight et al., Science 290:1972-1974 (2000); Ikeda et al., J Virol. 76:2997-3006 (2002); Lohmann et al., Science 285:110-113 (1999)). However, studies using parts of a virus can only give limited insights about the infectious process and pathogenesis.

Thus, for the development of effective therapies and for the production of protective vaccines, a system for the reproducible isolation of HCV from infected patients and the replication of infectious virus is needed. The present invention addresses these and other needs in the art.

### BRIEF SUMMARY OF THE INVENTION

The present invention provides methods and compositions for replicating infectious HCV in vitro.

One embodiment of the invention provides methods for replicating infectious hepatitis C virus (HCV) in vitro by: (a) contacting macrophages in vitro with a composition comprising HCV under conditions suitable for infection of the macrophages with HCV; (b) culturing the infected macrophages in vitro; (c) obtaining a culture supernatant comprising infectious HCV from the infected macrophages; (d) contacting non-macrophage cells with the culture supernatant under conditions suitable for infection of the non-macrophage cells with HCV; and (e) culturing the non macrophage, HCV-infected cells in vitro under conditions suitable for HCV production, thereby replicating infectious HCV in vitro. The macrophage and the non-macrophage cells may be human cells. The macrophages may be primary cells, cells isolated from fetal cord blood, and/or obtained by culturing mononuclear cells under conditions suitable for inducing differentiation of the mononuclear cells into macrophages. The composition comprising HCV may be, e.g., serum from an HCV-infected subject or peripheral blood mononuclear cells from an HCV-infected subject. The non-macrophage cells may be primary cells or immortalized cells and may be e.g., EBV-immortalized B cells, T cells, non-committed lymphoid cells, and neuronal precursor cells (e.g., metencephalon cells and telencephalon cells). In certain aspects, the HCV-infected cells of step (c) are passaged and produce infectious HCV for at least about 10, 15, 20, 23, 10, 50, 75, 100, 125, 150, 175, 200, 250, or 300 or more weeks.

Another embodiment of the invention provides methods for isolating infectious hepatitis C virus (HCV) particles from an in vitro culture by: (a) contacting macrophages with a composition comprising HCV under conditions suitable for infection of the macrophages with HCV; (b) culturing the infected macrophages in vitro; (c) obtaining culture supernatant comprising infectious HCV from the infected macrophages; (d) contacting non-macrophage cells with the culture supernatant under conditions suitable for infection of the cells with HCV; (e) culturing the HCV-infected non-macrophage cells under conditions suitable for HCV production; and (f) isolating HCV particles from culture supernatant of the HCV-infected non-macrophage cells. In some embodiments, the methods further comprise: (g) contacting different non-macrophage cells with the culture supernatant, thereby infecting the different non-macrophage cells with HCV in vitro.

A further embodiment of the invention provides methods of screening for compounds that inhibit of HCV production by: (a) contacting macrophages in vitro with a composition comprising HCV under conditions suitable for infection of the macrophages with HCV; (b) culturing the infected macrophages in vitro; (c) obtaining culture supernatant comprising infectious HCV from the infected macrophages; (d) contacting non-macrophage cells with the culture supernatant under conditions suitable for infection of the cell with HCV; (e) contacting the non-macrophage, HCV-infected cells with a compound suspected of having the ability to inhibit HCV production and culturing the HCV-infected cell under conditions suitable for HCV production; and (f) detecting the level of HCV production in the HCV-infected cell. A compound that decreases the level of HCV production in the HCV-infected cell relative to the level of HCV production in a HCV-infected cell that has not been contacted with the compound, is identified as a compound that inhibits HCV production. The compounds suspected of having the ability to inhibit HCV production may be e.g., interferons, agents that induces interferon-α production, CpG oligonucleotides, antisense oligonucleotides, agonists of toll-like receptor 9 (TLR9); protease inhibitors, and small organic compounds. The level of HCV production in the HCV-infected cell can be detected by detecting the presence of a HCV nucleotide or the presence of a HCV polypeptide. In some embodiments, the HCV nucleotide being detected hybridizes under stringent conditions with a oligonucleotide comprising the sequence set forth in SEQ ID NO:1. In some embodiments, the HCV nucleotide is detected by: (g) amplifying a HCV nucleotide from a culture supernatant from the HCV-infected cell of step (d) with a pair of oligonucleotide primers comprising the sequences set forth in SEQ ID NOS: 2 and 3 to obtain a first amplified product; (h) amplifying the first amplified product with a pair of oligonucleotide primers comprising the sequences set forth in SEQ ID NOS: 4 and 5 to obtain a second amplified product; and (i) detecting the second amplified product. In other embodiments, the HCV nucleotide is detected by: (e) amplifying a HCV nucleotide from a culture supernatant from the HCV-infected cell of step (d) with a pair of oligonucleotide primers comprising the sequences set forth in SEQ ID NOS: 6 and 7 to obtain a first amplified product; (f) amplifying the first amplified product with a pair of oligonucleotide primers comprising the sequences set forth in SEQ ID NOS: 8 and 9 to obtain a second amplified product; and (g) detecting the second amplified product.

Another embodiment of the invention provides stable cell in vitro cultures for long term replication of infectious HCV. In this aspect, the cells are HCV-infected non-macrophage cells obtained by contacting the non-macrophage cells with a culture supernatant from an in vitro cultured, HCV-infected macrophage and the HCV infected, non-macrophage cells produce infectious HCV. In certain aspects at least about 40%, 50%, 60%, 70%, 80%, or 90% or more of the cells in the culture produce infectious HCV.

A further embodiment of the invention provdes stable in vitro cell culture for long term replication of infectious HCV from a single patient isolate. In this aspect, the cells are HCV-infected non-macrophage cells obtained by contacting the non-macrophage cells with a culture supernatant from an in vitro cultured, HCV-infected macrophage and the HCV infected, non-macrophage cells produce infectious HCV. In certain aspects at least about 50%, 60%, 70%, 80%, or 90% or more of the cells in the culture produce infectious HCV.

An additional embodiment of the invention provides stable in vitro cell cultures for long term replication of infectious HCV. In this aspect, the cells are HCV-infected non-macrophage cells which produce infectious HCV. In certain aspects at least about 50%, 60%, 70%, 80%, or 90% or more of the cells in the culture produce infectious HCV.

Even another embodiment of the invention provides isolated nucleic acids comprising the sequence set forth in any one of SEQ ID NOS. 1-9.

Other embodiments and advantages of the present invention will be apparent from the detailed description that follows.

### BRIEF DESCRIPTION OF THE DRAWINGS

FIG. 1 illustrates isolation and replication of HCV from HCV-infected human patients. FIG. 1A illustrates the isolation scheme for the replication of HCV in vitro. FIG. 1B illustrates the history of transmission of the specimen donated from HCV infected patient #081. Fresh macrophages were infected by using cell-free serum or cocultured with HCV infected PBMC from the blood of patient #081. Human T-cells (112 A), B-cells (112 B) or the non-committed lymphoid cells (112 AB) were then either infected by cell-free transmission of HCV from cell culture supernatant from macrophages or cocultured with HCV infected macrophages. Similarly freshly transformed cord blood B-cells (PCLB 1°) were infected by cell free transmission from previously infected B-cell (112 B) culture supernatant. Uninfected transformed B-cells (PCLB T1-T4) were infected by serial, cell-free transmission from filtered PCLB 1° culture supernatant. Neuronal precursor cells were infected by cell free transmission of HCV from filtered #081 culture supernatant.

FIG. 2 illustrates data summarizing quantitation of molecules of positive-strand HCV-RNA per ml of cell culture supernatant via real-time RT-PCR.

FIG. 3 is Table 1 which sets forth sequences of primers used to analyze HCV. The primers were designed with the program PrimerSelect (DNASTAR) using conserved HCV sequences downloaded from GenBank.

FIG. 4 is Table 2 which summarizes the results of HCV transmission experiments with various hematopoetic and liver cells. (A) T-cells isolated from human fetal chord blood. (B) B-cells immortalized by infection with transforming EBV. (C) Monocyte/Macrophages, adherent cells stimulated with PMA. (D) Recently isolated neuronal cells from fetal brain. (E) Freshly isolated liver cells from liver biopsies. Kupffer's cells are liver macrophages and Hepatocytes are liver endothelial cells.

FIG. 5 is Table 3 which summarizes the history of HCV positivity for CIMM-HCV isolates in a variety of cell types. Each sample represents a monthly harvest of cell culture supernatants that were tested for the presence of human HCV positive-strand RNA and the cell free transmission to fresh target cells. Each individual sample was stored in liquid nitrogen at various time points throughout our testing. CIMM-HCV has been carried for over 12 months as a primary culture and over 31 months as a transmitted virus into other cell types including T-Cells (112A), B-Cells (112B), non-committed lymphoid cells (112AB) and 4th serial transmission into immortalized cord B-cells (PCLB T4). Primary cells are the first B-cells infected with HCV isolated from the macrophages.

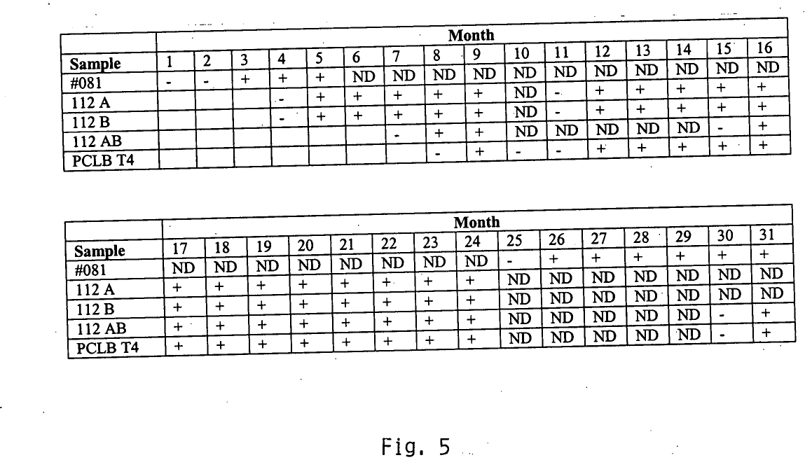

FIG. 6 is Table 4 which summarizes data from experiments demonstrating transmission of human HCV in neuronal precursor cells. The neuronal precursor cells were isolated from fetal brain. They were designated T (telencephalon, suspension cells) and M (metencephalon, adherent cells). Each sample represents a monthly harvest of cell culture supernatant that were tested for the presence of positive-strand HCV RNA.

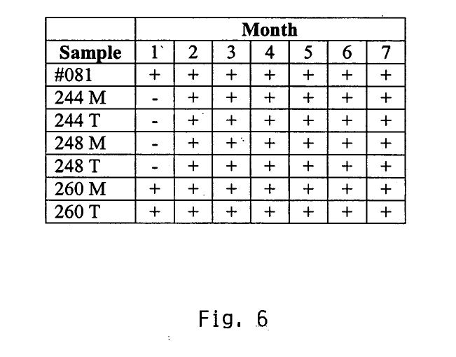

FIG. 7 is Table 5 which summarizes data from experiments that compared HCV primers known in the art (i.e., primers described in Chayama, Hepatitis C Protocols, J. Y. Lau Ed., vol 19 of Methods in Molecular Medicine (Humana Press, Totowa, N.J., 1998), pp. 165-173; Koylkhalov, et al., Hepatitis C Protocols, J. Y. Lau Ed., vol 19 of Methods in Molecular Medicine (Humana Press, Totowa, N.J., 1998), pp. 289-301; Norder et al., J. Clin. Micro. 36, 3066-3069 (1998); and Rispeter et al., J. Gen. Virol. 78, 2751-2759 (1997)). Primers described in Chayama, are labeled as (1); primers described in Koylkhalov, et al., are labeled as 2; primers described in Norder et al., are labeled as (3); primers described in Rispeter et al., are labeled as (4); primers that we developed are labeled as (5) to the HCV primers comprising sequences set forth in SEQ ID NOS: 2-9: (i)+=a positive PCR reaction, (ii)+=an RT-PCR reaction leading to HCV specific sequences. For either column, failure of the procedure is indicated by a negative sign (−).

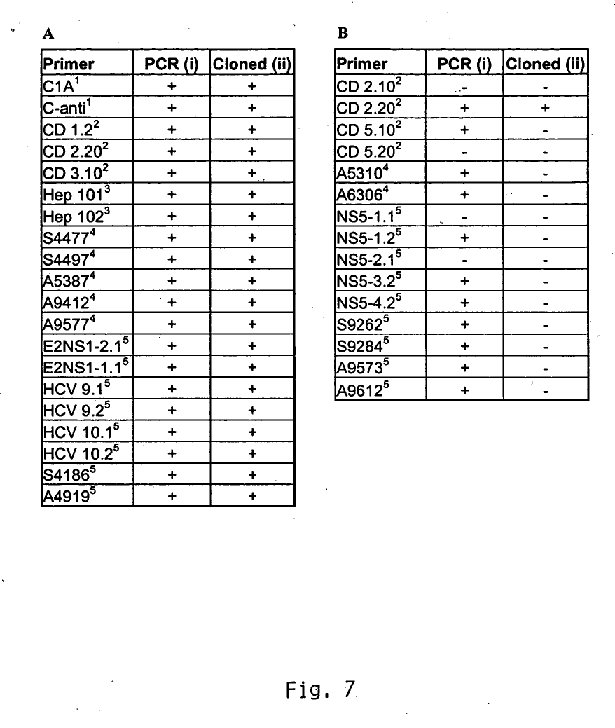

### DETAILED DESCRIPTION OF THE INVENTION

I. Introduction

The present invention provides methods for long term replication of infectious HCV iin vitro. The invention is based on the surprising discovery that macrophages can be used as an intermediate host cell to transmit infectious HCV to other cell types. Accordingly, in one embodiment, the invention provides a method for replicating infectious HCV by first propagating infectious HCV in macrophages and contacting the cell culture supernatant from the macrophages with a non-macrophage cell. The infectious HCV in the cell culture supernatant infects the non-macrophage cell which then produces infectious HCV. Cell culture supernatants from the HCV-infected non-macrophage cell can be used to infect other cells including, e.g., macrophage cells and non-macrophage cells, with HCV. As set forth in the Examples below, the HCV-infected non-macrophage cells can be stable in vitro cell cultures for replication of infectious HCV (e.g., from a single patient isolate). Using these methods and cell cultures, infectious HCV can be replicated in vitro on a short term, medium term and long term basis in multiple cell types.

In some embodiments, the HCV-infected cell cultures and infectious HCV isolated from the cell culture supernatants can be used to develop additional diagnostics and therapeutics for treating HCV (e.g., in in vitro assays to identify inhibitors of infectious HCV production). In some aspects, the invention provides HCV isolate specific primers and probes which can be used to detect different HCV isolates. The specific HCV isolates can in turn be used to develop diagnostics and therapeutics directed toward specific HCV strains.

II. Definitions

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described below are those well known and commonly employed in the art. Standard techniques are used for nucleic acid and peptide synthesis. Generally, enzymatic reactions and purification steps are performed according to the manufacturer's specifications. The techniques and procedures are generally performed according to conventional methods in the art and various general references (see generally, Sambrook et al. MOLECULAR CLONING: A LABORATORY MANUAL, 3d ed. (2001) Cold Spring Harbor Laboratory Press, Cold Spring Harbor, N.Y.), which are provided throughout this document. The nomenclature used herein and the laboratory procedures in analytical chemistry, and organic synthetic described below are those well known and commonly employed in the art. Standard techniques, or modifications thereof, are used for chemical syntheses and chemical analyses.

“Hepatitis C virus” or “HCV” is a linear positive-sense single stranded RNA virus of with a genome of about 10,000 nucleotides in length that encodes a polyprotein of about 3000 amino acids. Multiple HCV isotypes have been identified including HCV 1a. These and other isotypes are described in e.g., Blight et al., J. Virol. 77(5):3181-3190 (2003); U.S. Pat. Nos. 5,585,258; 5,670,152; 5,670,153; 5,683,864; 5,714,596; and 5,728,520. HCV nucleotide sequences are set forth in the following Genbank Accession Nos.: M62321; M58406; M58407; D90208; and M58335. HCV nucleotide sequences include the 5′UTR region of the HCV genome and sequences that hybridize under stringent conditions to the sequence set forth in SEQ ID NO:1. HCV poplypeptides and isotypes are described in, e.g., U.S. Pat. Nos. 5,585,258; 5,670,152; 5,670,153; 5,683,864; 5,714,596; and 5,728,520. The HCV genome encodes a polyprotein which is subsequently processed into a number of mature structural and nonstructural moieties (see, Grakoui et al., J. Virol. 67:2832-2843 (1993)). The host cell signal peptidases cleave the N-terminal region of the precursor polypeptide to produce the HCV core protein (see, Harada et al., J. Virol. 65:3015-3021 (1991); Hijikata et al., PNAS USA. 88:5547-5551 (1991); and Selby et al., J Gen Virol. 74:1103-1113 (1993)). The HCV core protein is reported to range between 16 and 25 kDa in size (see, Hijikata et al., PNAS USA. 88:5547-5551 (1991); Lo et al., Virology 199:124-131 (1994); Yasui et al., J. Virol. 72:6048-6055 (1998); and Yeh et al., J Gastroenterol Hepatol. 15:182-191 (2000)).

As used herein, the term “infectious HCV” refers to an HCV particle which can infect a cell, e.g., a macrophage or a non-macrophage cell including, e.g., B cell, T cell, a non-committed lymphoid cell, or a neuronal precursor cell (e.g., a telencephalon cell or a metencephalon cell) and replicate within the cell such that the cell produces infectious HCV particles.

“Macrophages” are terminally differentiated cells that originate from a precursor stem cell found in bone marrow. This stem cell is thought to be a common multipotential stem cell that eventually leads to all the cells of the hematolymphoid system (see, e.g., Fundamental Immunology (Paul ed., 3d ed. (1993); Immunology (Hood et al., eds., 2d ed. 1984)). Within particular maturational microenvironments, this multipotential stem cell develops into a myeloid stem cell, and then commits to a specific developmental lineage (see, e.g., Paul, supra, for a discussion of proteins involved in monocyte-derived macrophage differentiation). Developmental commitment to the macrophage lineage is demonstrated by the monocyte, which is a differentiated precursor of a macrophage. Monocytes are found circulating in the blood, in tissues, and in a storage compartment presumably located in the bone marrow. In tissues, monocytes develop further into macrophages. Under normal circumstances, neither monocytes or macrophages divide.

Macrophages are found in all tissues, in surrounding blood vessels, and close to epithelial cells. Macrophages in different tissues can develop distinctive properties. For example, macrophages from peritoneal cavity, lung, liver, kidney, bone marrow, and spleen have different cell receptors, expression of MHC class II molecules, and oxidative metabolism. Their main function is to investigate the environment, respond to stimuli, and present antigen via MHC class II. Therefore, macrophages are active in pinocytosis, where they sample extracellular fluid, and they also express surface receptors to a wide range of molecules. In this manner, macrophages can take up microorganisms and respond to cytokines and foreign proteins. In response to these environmental stimuli, the macrophages present internalized antigen to other cells of the immune system, and they secrete a variety of molecules. Thus, macrophages participate in inflammation and immunological reactions, such as antigen presentation to T cells via MHC class II molecules.

“Monocyte” refers to a differentiated cell of the mononuclear phagocyte lineage, e.g., those that are CD14⁺ (see, e.g., Fundamental Immunology (Paul ed., 4th ed. 1999)). “Monocyte-derived macrophage” or “MDM” is a type of antigen presenting cell of the mononuclear phagocyte lineage derived from monocytes that have further differentiated into macrophages (see, e.g., Paul, supra).

“Non-macrophage cells” include any cells that have not terminally differentiated into a macrophage. Accordingly, non-macrophage cells include differentiated macrophage precursor cells such as monocytes. Additional non-macrophage cells suitable for use in the methods of the invention include, e.g., B cells (primary and immortalized, including EBV-immortalized B cells), T cells, noncommitted lymphoid cells, liver cells, and neuronal precursor cells from the telencephalon (e.g., BF-1⁺ cells as described in, e.g., Chun and Jaenisch, Mol Cell Neurosci. 7(4):304-21 (1996)) and metancephalon (e.g., Hoxb-1⁺, Fgf3⁺, and/or MafB⁺ cells as described in, e.g., Gale et al., Mech Dev. 1999 December; 89(1-2):43-54 (1999)). Non-macrophage cells may be freshly isolated from tissues, whole blood, or cord blood; or may be maintained in culture. For example, telencephalon cells can be isolated from the anterior portions of the brain, e.g., the cerbral cortex, basal ganglia, corpus striatum, and olfactory bulb and metencephalon cells can be isolated from the hindbrain, e.g., the pons and the cerebellum.

“Peripheral blood mononuclear cells” or “PBMC” refers to a heterogeneous population of hematolymphoid cells derived from blood, from which the red blood cells have been removed.

“Cord blood mononuclear cells” or “CBMC” refers to a heterogeneous population of hematolymphoid cells derived from cord blood, from which the red blood cells have been removed.

As used herein the term “HCV production” refers to production of infectious HCV particles by serial passage of infectious HCV particles from one cell culture to another under conditions such that the cell cultures are HCV positive (i.e., HCV nucleotides or polypeptides can be detected in the cell cultures). The infectious HCV particles may be passaged by contacting cell culture supernatant from HCV-infected cells with uninfected cells or by coculturing HCV-infected cells with uninfected cells. The cells may be the same type or a different type. Production of HCV particles includes any of the steps the HCV life cycle including replication of the HCV viral genome, transcription, translation, and HCV particle assembly and release. “Short term production” of HCV refers to production of HCV particles by serial passage of infectious HCV particles from one cell culture to another for 0 to about 10 weeks. “Medium term production” of HCV refers to production of HCV particles by serial passage of infectious HCV particles from one cell culture to another for about 10 to about 23 weeks. “Long term production” of HCV refers to production of HCV particles by serial passage of infectious HCV particles from one cell culture to another for about 23, 30, 40, 50, 75, 100, 125, 130, 150, 175, 200, 225, 250, 275, 300, or more weeks.

An “inhibitor of HCV production” may inhibit one or more of the steps involved in HCV life cycle. An inhibitor of HCV production may also inhibit attachment of the HCV to its target cell, i.e., a liver cell. Inhibitors of HCV production include, for example, interferons and their analogues and compounds that induce interferon production. Typically an inhibitor of HCV production causes at least about a 10%, 20%, 30%, 40%, 50%, 60%, 70% 80% or 90% decrease in the HCV viral titer (i.e., number of viral particles) in a cell culture supernatant relative to the HCV viral titer in a cell culture supernatant from cells that have not been contacted with the inhibitor. Potential inhibitors of HCV production include, e.g., interferons (e.g., interferon α or interferon γ), agents that induce interferon production (e.g., 2-amino-8-hydroxyadenines), oligonucleotides including CpG-containing oligonucleotides, antisense oligonucleotides, and siRNA; proteins longer nucleic acids, and small organic molecules.

“Culturing” refers to growing cells ex vivo or in vitro. A “stable cell culture” refers to a culture of cells, typically non-macrophage cells (e.g., fresh B cells, EBV immortalized B cells, noncommitted lymphoid cells, or neuronal precursor cells) which retain the ability to produce HCV particles, e.g., into the cell supernatant through multiple passages (e.g., over a period of 2, 5, 10, 15, 20, 23, or more weeks). Typically at least about 60%, 70%, 80%, or 90% of the cells in a stable cell culture produce HCV particles. The stable cell cultures may comprise one or more different cell types. The stable cell culture may comprise infectious HCV from a single HCV-infected individual (i.e., a single patient isolate) or from multiple HCV-infected individuals. Stable cell cultures include cell cultures that retain the ability to produce HCV particles through multiple freeze/thaw cycles.

The term “antibody” refers to a polypeptide encoded by an immunoglobulin gene or functional fragments thereof that specifically binds and recognizes an antigen (e.g., an HCV polypeptides including envelope glycoproteins E1 and E2; nonstructural proteins NS1 and NS2, and HCV core antigen).

The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist e.g., as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C._{H}1 by a disulfide bond. The F(ab)₂ may be reduced under mild conditions to break the disulfide linkage in the hinge region, thereby converting the F(ab)′₂ dimer into an Fab′ monomer. The Fab′ monomer is essentially an Fab with part of the hinge region (see Fundamental immunology (Paul, ed., 4th ed. 1999)). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies or those synthesized de novo using recombinant DNA methodologies (e.g., single chain Fv).

The term “immunoassay” is an assay that uses an antibody to specifically bind an analyte. The immunoassay is characterized by the use of specific binding properties of a particular antibody to isolate, target, and/or quantify the analyte.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. In particular, an isolated HCV nucleic acid is separated from the HCV viral particle. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The terms “nucleic acid” and “polynucleotide” are used interchangeably herein to refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic. Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins (1984)).

“Amplifying” refers to submitting a solution to conditions sufficient to allow for amplification of a target polynucleotide i.e., an HCV sequence) if all of the components of the reaction are intact. Components of an amplification reaction include, e.g., primers, a polynucleotide template, polymerase, nucleotides, and the like.

The term “subsequence” refers to a sequence of nucleotides that are contiguous within a second sequence but does not include all of the nucleotides of the second sequence.

A “target” or “target sequence” refers to a single or double stranded polynucleotide sequence sought to be amplified in an amplification reaction. Two target sequences are different if they comprise non-identical polynucleotide sequences.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, preferably 65%, 70%, 75%, 80%, 85%, 90%, or 95% identity over a specified region such as the 5′UTR of the HCV genome), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Preferably, the identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins to HCV nucleic acids and proteins, the BLAST and BLAST 2.0 algorithms and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4 and a comparison of both strands. For amino acid sequences, the BLASTP program uses as defaults a word length of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence (e.g., a HCV sequence set forth in SEQ ID NO:1) under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For high stringency hybridization, a positive signal is at least two times background, preferably 10 times background hybridization. Exemplary high stringency or stringent hybridization conditions include: 50% formamide, 5×SSC and 1% SDS incubated at 42° C. or 5×SSC and 1% SDS incubated at 65° C., with a wash in 0.2×SSC and 0.1% SDS at 65° C.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min.

The phrase “selectively associates with” refers to the ability of a nucleic acid to “selectively hybridize” with another as defined above.

“Biological sample” as used herein is a sample of biological tissue or fluid that is suspected of containing a nucleic acid encoding a HCV polypeptide or a HCV polypeptide. These samples can be tested by the methods described herein and include cell culture supernatants, cells maintained in culture as well as body fluids such as whole blood and blood fractions including, e.g., serum, plasma, lymph fluids, and lymphocytes, and the like; and tissue samples including liver samples. The samples may be fresh, frozen, or preserved in a fixative such as paraffin. A biological sample is obtained from any mammal including, e.g., primates such as humans and chimpanzees. A biological sample may be suspended or dissolved in liquid materials such as buffers, extractants, solvents and the like.

III. Methods of Replicating Hepatitis C Virus In Vitro

The present invention provides methods and compositions for replication of HCV in vitro. According to the methods of the inventions, the infectious HCV is first propagated in intermediate host cells (i.e., a macrophages) by contacting the macrophages with a composition comprising HCV and culturing the host cell in vitro. Once HCV has been cultured in the host cell, HCV-containing cell culture supernatant from the host cell is contacted with a second cell, i.e., a non-macrophage, and cultured for replication (i.e., long term, medium term, or short term) of infectious HCV in vitro. Preferably the macrophages and non-macrophage cells are all human cells. In some embodiments, HCV-containing cell culture supernatant from the second cell is contacted with another non-macrophage cell for further replication of infectious HCV in vitro. The second and third non-macrophage cells may be the same cell type or different cell types.

The intermediate host cells and the cells for production of infectious HCV may be derived from any suitable mammal. For example the cells may be obtained from primates such as, for example, chimpanzees and humans, rodents such as, for example, mice, rats, guinea pigs, and rabbits; and non-rodent mammals such as, for example, dogs, cats, pigs, sheep, horses, cows, and goats. Preferably the cells are human cells. The cells to be cultured may be primary cells or may be cells maintained in culture. Techniques and methods for establishing a primary culture of cells and for maintaining cells in culture for use in the methods of the invention are known to those of skill in the art. See e.g., Humason, ANIMAL TISSUE TECHNIQUES, 4^{th} ed., W. H. Freeman and Company (1979), and Ricciardelli et al., (1989) In Vitro Cell Dev. Biol. 25: 1016.

A. Intermediate Host Cells

Typically, macrophages are used as the intermediate host cells. The macrophages used as intermediate host cells may be isolated from any source (e.g., whole blood, liver tissue) using methods known in the art. In a preferred embodiment, macrophages are isolated by stimulating mononuclear cells to differentiate into macrophages. The mononuclear cells can be isolated from any source known in the art including, e.g., whole blood (i.e., PBMC), bone marrow, and cord blood (i.e., CBMC). In a preferred embodiment, the mononuclear cells are isolated from cord blood, typically human cord blood. CBMC and PBMC are prepared from cord blood samples and whole blood samples, respectively, by separating mononuclear cells from red blood cells. There are a number of methods for isolating CBMC and PBMC, e.g., velocity sedimentation, isotonic sedimentation, affinity purification, and flow cytometry. Typically, CBMC and PBMC are separated from red blood cells by density gradient (isotonic) centrifugation, in which the cells sediment to an equilibrium position in the solution equivalent to their own density. For density gradient centrifugation, physiological media should be used, the density of the solution should be high, and the media should exert little osmotic pressure. Density gradient centrifugation uses solutions such as sodium ditrizoate-polysucrose, Ficoll, dextran, and Percoll (see, e.g., Freshney, Culture of Animal Cells (3rd ed. 1994)). Such solutions are commercially available, e.g., HISTOPAQUE™ (Sigma). Typically, anticoagulated whole blood or cord blood is layered onto the gradient and centrifuged according to standard procedures (see, e.g., Fish et al., J. Virol. 69:3737-3743 (1995)). Using, e.g., the procedure in Fish et al., the red blood cells and granulocytes form a pellet, while lymphocytes and other mononuclear cells such as monocytes remain at the plasma/density gradient interface (see, e.g., Freshney, Culture of Animal Cells (3rd ed. 1994)).

Once the CBMC and PCMC have been isolated, they are cultured under conditions that give rise to differentiation of mononuclear cells into macrophages. In a preferred embodiment, treatment with Phorbol-12-myristate-13-acetate (PMA) at about 5 ng/ml in the culture medium) as described in, e.g., Salahuddin et al., Science 242:430-433 (1988) is used to induce differentitation of mononuclear cells into macrophages that can serve as intermediate host cell. The cells are typically maintained in media that is composed of approximately 50% spent media:50% fresh media. The media is replenished approximately every 3-4 days, and the cultures can be maintained for at least about 2, 4, 6, 8, 10, or more weeks.

Macrophages can also be derived from mononuclear cells using allogeneic stimulation as described in U.S. Pat. No. 6,225,408. The cells that are subjected to allogeneic stimulation are isolated from any suitable source and may be heterogenous or homogenous, e.g., peripheral blood mononuclear cells (“PBMC”) or monocytes. For cell-mediated allogeneic stimulation reactions, PBMC are typically used as the source of the monocyte-derived macrophage cultures of the invention. Monocytes can be quickly isolated from PBMC with a 2 hour adherence onto plastic. After the cells of choice are isolated, they are cultured under conditions that give rise to differentiation of monocytes into monocyte-derived macrophages (“MDM”). For example, the MDM can be cultured under conditions where monocytes are separated from allogeneically stimulated PBMC, using semipermeable membranes. The MDM can also be generated using a cell free, cytokine-mediated allogeneic stimulation reaction, PBMC or monocytes from a single individual are directly treated with cytokines such as IFN-γ to activate differentiation of monocytes into MDM.

Once the macrophages have been isolated, they can be contacted with compositions comprising infectious HCV and cultured using methods known in the art. Cell culture supernatants from the HCV-infected macrophages can be used to infect other cells (i.e., nonmacrophage cells) with HCV or may be used as a source of infectious HCV particles.

Suitable cell culture methods and conditions can be determined by those of skill in the art using known methodology (see, e.g., Freshney et al., CULTURE OF ANIMAL CELLS (3rd ed. 1994)). In general, the cell culture environment includes consideration of such factors as the substrate for cell growth, cell density and cell contract, the gas phase, the medium, and temperature.

Typically plastic dishes or flasks are used. Other artificial substrates can be used such as glass and metals. The substrate is often treated by etching, or by coating with substances such as collagen, chondronectin, fibronectin, and laminin. The type of culture vessel depends on the culture conditions, e.g., multi-well plates, petri dishes, tissue culture tubes, flasks, and the like. Cells are grown at optimal densities that are determined empirically based on the cell type. For example, before adherence, a typical cell density for mononuclear cell cultures varies from about 1×10⁶ to about 1×10⁸ per ml of medium, and after adherence the typical cell density is about 1×10⁴ to about 1×10⁶ cells per ml.

Important constituents of the gas phase are oxygen and carbon dioxide. Typically, atmospheric oxygen tensions are used for the cultures. Culture vessels are usually vented into the incubator atmosphere to allow gas exchange by using gas permeable caps or by preventing sealing of the culture vessels. Carbon dioxide plays a role in pH stabilization, along with buffer in the cell media and is typically present at a concentration of 1-10% in the incubator. The preferred CO₂ concentration is 5%.

Cultured cells are normally grown in an incubator that provides a suitable temperature, e.g., the body temperature of the animal from which is the cells were obtained, accounting for regional variations in temperature. Generally, 37° C. is the preferred temperature for cell culture. Most incubators are humidified to approximately atmospheric conditions.

Defined cell media are available as packaged, premixed powders or presterilized solutions. Examples of commonly used media include Iscove's media, AIM-V, RPMI 1640, DMEM, and McCoy's Medium (see, e.g., GibcoBRL/Life Technologies Catalogue and Reference Guide; Sigma Catalogue). Defined cell culture media are often supplemented with 5-20% serum, e.g., human horse, calf, and fetal bovine serum. Preferably the serum is 10% non-heat inactivated human serum (Sigma). The culture medium is usually buffered to maintain the cells at a pH preferably from 7.2-7.4. Other supplements to the media include, e.g., antibiotics, amino acids, sugars, and growth factors.

B. Compositions Comprising HCV

Any source of HCV can be used to infect the macrophage host cells. Suitable sources of HCV include, e.g., serum from an HCV-infected individual (e.g., a human), peripheral blood mononuclear cells (“PBMC”) from an HCV-infected individual; and culture supernatant from HCV-infected cells.

Serum can be prepared from fresh or frozen whole blood (i.e., from HCV-infected individuals) using methods known in the art. Typically, whole blood is collected from and HCV-infected individual and allowed to clot. The clotted blood is centrifuged for about 10 min at 1500 rpm in a standard centrifuge (e.g., a Sorvall RC-3B) and the supernatant (i.e., HCV-containing serum) is collected. The serum may be collected from a single HCV-infected individual (i.e., to generate a single patient isolate of HCV) or from multiple HCV-infected individuals.

HCV-infected PBMC are prepared from whole blood samples (i.e., from HCV infected individuals) by separating mononuclear cells from red blood cells as described above. Once the HCV-infected PBMC have been prepared, they are cocultured with macrophages under conditions such that the macrophages are infected with the HCV. Typically, the HCV-infected PBMC are mixed approximately 1:1 with macrophages and the two cells types are cocultured at 37° C. in a 5% CO₂ atmosphere. After about 24 hours, the media is changed and the cells are cultured for about another 6 days, with a change of media on day 4. The cells are typically maintained in media that is composed of approximately 50% spent media:50% fresh media. The media is replenished approximately every 3-4 days, and the cultures can be maintained for at least about 2, 4, 6, 8, 10, or more weeks.

HCV-containing cell culture supernatant is obtained by apirating culture media from a culture of HCV-producing cells that have been cultured for at least about 24, 48, 72, or more hours. To obtain a cell free HCV-containing culture supernatant, the media is passed through a 0.45 μm filter.

HCV viral particles can be isolated from the serum of HCV-infected individuals and from the cell culture supernatant of HCV-infected cells. Methods of isolating viral particles are well known in the art and include gradient centrifugation and flow filtration (see, e.g., Anderson et al., Emerging Infect. Diseases 9(7): 768-773 (2003)). Isolated HCV viral particles can be used to, e.g., infect cell cultures for production of infectious HCV and in studies to further characterize particular HCV isolates.

C. Cells for HCV Production

Any non-macrophage cell known in the art can be used for production of infectious HCV. For example, T cells, B cells, noncommitted lymphoid cells (e.g., pluripotent hematopoietic cells), and neuronal precursor cells can be used for production of infectious HCV. The HCV-producing cell culture may comprise one cell type or multiple cell types. Suitable cells include primary cells and immortalized cells. Preferably the cells are human cells. In preferred embodiments, B cells and neuronal precursor cells (e.g., cells derived from the telencephalon and metancephalon,) are used for long term production (i.e., 23 weeks or more) of infectious HCV. In a particularly preferred embodiment, Epstein-Barr virus (EBV)-immortalized B cells are used.

Methods of immortalizing B cells with EBV are well known in the art and are described in, e.g., Roth et al., Blood 84(2): 582-587 (1994). B cells can be obtained from any source including, e.g., whole blood, lymph nodes, spleen. B cells can also be obtained by culturing precursor cells (e.g., from bone marrow or cord blood) under conditions that give rise to B cells. In a preferred embodiment, B cells to be immortalized are generated by culturing cord blood mononuclear cells under conditions that give rise to B cells. Typically, the CBMCs are stimulated with pokeweed mitogen (PWM) at about 5 μg/ml. The PWM-induced B cells are then infected by contact with transforming Epstein-Barr virus (EBV) as described in, e.g., Roth et al., supra and Fingeroth et al., PNAS USA 81:4510-4514 (1984). Typically about 10×10⁶ PWM induced B cells are incubated in 2 ml EBV containing cell culture supernatant for about 2 hours at 37° C. in 5% CO₂. Cyclosporin A is then added to a final concentration of about 0.1 μg/ml and the cells are incubated at 37° C. in 5% CO₂ for 2-3 weeks. The immortalized B cells can then be used for production of infectious HCV. Any strain of transforming EBV known in the art can be used to immortalize the B cells so long as transformation with the strain does not induce the transformed B cell to produce EBV.

Additional suitable non-macrophage cell for infectious HCV production can be isolated and cultured using methods known in the art (see, e.g., U.S. Pat. No. 6,610,540; U.S. Patent Publication Nos. 2004000504 and 20030211605; and Freshney et al. supra). For example neuronal precursor cells can be isolated from the anterior or posterior sections of the brain. In particular, telencephalon cells can be isolated from the anterior portions of the brain, e.g., the cerbral cortex, basal ganglia, corpus striatum, and olfactory bulb and metencephalon cells can be isolated from the hindbrain, e.g., the pons and the cerebellum Once isolated, the cells are typically cultured on a substrate-coated surface. Suitable substrates include, e.g., poly-L-lysine and polyethyleneimine. Neuronal precursor cells can also be cultured from embryonic stem cells as described in, e.g., U.S. Patent Publication Nos. 20030211605. In additional, neuronal cell lines can be obtained from commercial sources.

IV. Detection of Infectious HCV in Cell Cultures

Infectious HCV can be detected in cell cultures using a variety of techniques known in the art. HCV genomes and transcripts are detected using nucleic acid hybridization and amplification techniques. HCV particles and proteins are detected using any one of a number of immunological techniques known in the art. HCV titer and infectivity is confirmed by isolating supernatants from HCV-infected cells and then infecting other cells with HCV. The methods of detecting infectious HCV described herein can conveniently be used to determine whether a cell culture is producing infectious HCV particles, to diagnose HCV infection in an individual suspected of being infected with HCV, to identify the specific HCV strain infecting an individual, and to develop therapeutics for treating HCV.

A. Hybridization Assays to Detect HCV Nucleotides

Techniques used to detect HCV nucleotides include a variety of techniques based on nucleic acid hybridization, e.g. Northern blots, Southern blots, and dot blots. Suitable nucleic acid hybridization techniques include those based on nucleic acid amplification as described herein.

Nucleic acid primers and probes used for hybridization assays are chosen to hybridize to a target HCV nucleotide. Hybridization conditions are selected by those of skill in the art. Although primers and probes can differ in sequence and length, the primary differentiating factor is one of function: primers typically serve as an initiation point for DNA synthesis of a target sequence (e.g., in an RT-PCR reaction) and probes are typically used for hybridization to and detection of a complementary target nucleic acid.

In a preferred embodiment, in situ hybridization can be used to detect HCV nucleotides. Methods of in situ hybridization are well known in the art and are described in, e.g.,: Tautz and Pfeifle, Chromosoma 98(2):81-5 (1989) and Sambrook et al., supra). Briefly, HCV-infected cells are fixed on a solid support, e.g., a glass slide and treated with proteases to degrade cellular proteins. An oligonucleotide probe (e.g., comprising the sequence set forth in SEQ ID NO:1 or a subsequence thereof) is hybridized to the HCV nucleic acids on the support and the presence of the hybridized probe is detected. In a preferred embodiment, the probe comprises the sequence set forth in SEQ ID NO:1.

One preferred hybridization assay is reverse transcription. Reverse transcription is an amplification method that copies RNA into DNA. The reverse transcription reaction, which synthesizes first strand cDNA, is typically performed by mixing HCV RNA with random hexamer primer or a specific primer (e.g., a primer comprising a sequence set forth in SEQ ID NOS: 2-9), heating to 70° C. for 5 minutes to denature the nucleic acids (a thermal cycler may be used for this step), and then cooling on ice. The reaction mixture, prepared according to the enzyme manufacturers instructions or according to kit instructions, is added to the denatured RNA and hexamer mixture and incubated at a suitable temperature, usually 42° C. The reaction is stopped by heating the tube containing the reaction mixture for 10 minutes at 70° C. The first strand cDNA is collected by precipitation and brief centrifugation and aliquoted to new tubes, in which it can be quickly frozen on dry ice and stored at −70° C., if necessary, for later use.

A final preferred method of detecting the presence of viral genome sequences is Northern hybridization. Briefly, RNA is isolated from a HCV-infected cell. The isolated RNA is run on agarose slab gels in buffer and transferred to membranes. Hybridization to the membrane is carried out using labelled probes which specifically hybridize to HCV nucleic acids (e.g. probes comprising the sequence set forth in SEQ ID NO:1) (see, e.g., Ausubel et al., supra; Sambrook et al., supra).

B. Amplification to Detect HCV Nucleotides

In some embodiments, amplification based detection methods are used to detect HCV nucleotides. Typically RT-PCR is used to detect HCV nucleotides. RT-PCR permits the copying, and resultant amplification of a target nucleic acid, e.g., an HCV nucleotide from the 5′UTR. In RT-PCR, an HCV RNA sequence is first reverse transcribed into a HCV cDNA sequence which is then amplified as described herein. The amplified HCV sequence is then detected using methods known in the art.

Amplification of a RNA or DNA template using PCR reactions is well known (see, U.S. Pat. Nos. 4,683,195 and 4,683,202; PCR Protocols: A GUIDE TO METHODS AND APPLICATIONS (Innis et al., eds, 1990); and PCR Technology: Principles and Applications for DNA Amplification (Erlich, ed. (1992)) Exemplary PCR reaction conditions typically comprise either two or three step cycles. Two step cycles have a denaturation step followed by a hybridization/elongation step. Three step cycles comprise a denaturation step followed by a hybridization step followed by a separate elongation step. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 15 seconds-2 minutes, an annealing phase lasting 10 seconds.-2 minutes, and an extension phase of about 72° C. for 5 seconds-2 minutes.

In general, PCR and other methods of amplification use primers which anneal to either end of the HCV 5′UTR. For example, the HCV 5′UTR may be amplified using isolated nucleic acid primer pairs comprising the sequences set forth in Table 1.

Typical RT-PCR reaction components include, e.g., a target sequence, reverse transcriptase, oligonucleotide primers, oligonucleotide probes, buffers (e.g., borate, phosphate, carbonate, barbital, Tris, etc. based buffers), salts (e.g., NaCl or KCl), a source of magnesium ions, dNTP's, and a nucleic acid polymerase (e.g., Taq DNA polymerase). PCR reactions can also include additional agents such as DMSO and stabilizing agents (e.g., gelatin, bovine serum albumin, and non-ionic detergents (e.g. Tween-20)).

The oligonucleotides (i.e., primers and probes) can be prepared by any suitable method, including chemical synthesis. Alternatively, they can be purchased through commercial sources. Methods of synthesizing oligonucleotides are well known in the art (see, e.g, Narang et al., Meth. Enzymol. 68:90-99, 1979; Brown et al., Meth. Enzymol. 68:109-151, 1979; Beaucage et al., Tetrahedron Lett. 22:1859-1862, 1981; and the solid support method of U.S. Pat. No. 4,458,066). These oligonucleotides can be labeled with radioisotopes, chemiluminescent moieties, or fluorescent moieties. Such labels are useful for the characterization and detection of amplification products using the methods and compositions of the present invention.

The primers are typically about 15 to about 60 nucleotides in length and are typically present in the PCR reaction mixture at a concentration of between about 0.1 and about 1.0 μM or about about 0.1 to about 0.75 μM. Typically the magnesium ion is present at about a 0.5 to 2.5 mM excess over the concentration of deoxynucleotide triphosphates (dNTPs). dNTPs typically are added to the reaction to a final concentration of about 20 μM to about 300 μM. Typically, each of the four dNTPs (G, A, C, T) are present at equivalent concentrations. (See, Innis et al.).

A variety of DNA dependent polymerases are commercially available that will function using the methods and compositions of the present invention. For example, Taq DNA Polymerase may be used to amplify the HCV sDNA sequences. Taq DNA polymerase which may be the native enzyme purified from Thermus aquaticus and/or a genetically engineered form of the enzyme. Other commercially available polymerase enzymes include, e.g., Taq polymerases marketed by Promega or Pharmacia. Other examples of thermostable DNA polymerases that could be used in the invention include DNA polymerases obtained from, e.g., Thermus and Pyrococcus species. Concentration ranges of the polymerase may range from 1-5 units per reaction mixture. The reaction mixture is typically between 20 and 100 μl.

One of skill in the art will recognize that buffer conditions, salt concentrations, magnesium ion concentrations, and dNTP concentrations can be designed to allow for the function of all reactions of interest, i.e., to support the amplification reaction as well as any subsequent restriction enzyme reactions. A particular set of reaction components can be tested for its ability to support various reactions by testing the components both individually and in combination. The optimal reaction conditions can vary depending on the nature of the target nucleic acid(s) and the primers being used, among other parameters.

In some embodiments, a “hot start” polymerase can be used to prevent extension of mispriming events as the temperature of a reaction initially increases. Hot start polymerases can have, for example, heat labile adducts requiring a heat activation step (typically 95° C. for approximately 10-15 minutes) or can have an antibody associated with the polymerase to prevent activation.

In some embodiments, the amplification reaction is a nested PCR assay as described in, e.g., Gonzalez-Perez et al., Biologicals 31(1):55-61 (2003). Two amplification steps are carried out. The first amplification uses an “outer” pair of primers (e.g., SEQ ID NOS: 2 and 3 or 6 and 7) designed to amplify a highly conserved region of the target sequence. The second amplification uses an “inner” (i.e., “nested”) pair of primers (e.g., SEQ ID NOS: 4 and 5 and 8 and 9) designed to amplify a portion of the target sequence that is contained within the first amplification product. Typically, forty cycles of amplification are performed with the following temperature profiles: 94° C. for 1 min, 55° C. for 1 min, and 72° C. for 1 min for the outer primer set and 94° C. for 1 min, 60° C. for 1 min, and 72° C. for 1 min for the inner primer set.

Isothermic amplification reactions are also known and can be used according to the methods of the invention. Examples of isothermic amplification reactions include strand displacement amplification (SDA) (Walker, et al. Nucleic Acids Res. 20(7):1691 (1992); Walker PCR Methods Appl 3(1): 1(1993)), transcription-mediated amplification (Phyffer, et al., J. Clin. Microbiol. 34:834 (1996); Vuorinen, et al., J. Clin. Microbiol. 33:1856 (1995)), nucleic acid sequence-based amplification (NASBA) (Compton, Nature 350(6313):91 (1991), and branched DNA signal amplification (bDNA) (see, e.g., Iqbal et al., Mol. Cell Probes 13(4):315 (1999)). In a preferred embodiment, rolling circle amplification (RCA) (Lisby, Mol. Biotechnol. 12(1):75 (1999)); Hatch et al., Genet. Anal. 15(2):35 (1999)) is used. Other amplification methods known to those of skill in the art include CPR (Cycling Probe Reaction), SSR (Self-Sustained Sequence Replication), SDA (Strand Displacement Amplification), QBR (Q-Beta Replicase), Re-AMP (formerly RAMP), RCR (Repair Chain Reaction), TAS (Transcription Based Amplification System), and HCS (hybrid capture system).

C. Detection of Amplified Products

Any method known in the art can be used to detect the amplified products, including, for example solid phase assays, anion exchange high-performance liquid chromatography, and fluorescence labeling of amplified nucleic acids (see MOLECULAR CLONING: A LABORATORY MANUAL (Sambrook et al. eds. 3d ed. 2001); Reischl and Kochanowski, Mol. Biotechnol. 3(1): 55-71 (1995)). Gel electrophoresis of the amplified product can also be used to detect and quantify the amplified product. Suitable gel electrophoresis-based techniques include, for example, gel electrophoresis followed by quantification of the amplified product on a fluorescent automated DNA sequencer (see, e.g., Porcher et al., Biotechniques 13(1): 106-14 (1992)); fluorometry (see, e.g., Innis et al., supra), computer analysis of images of gels stained in intercalating dyes (see, e.g., Schneeberger et al., PCR Methods Appl. 4(4): 234-8 (1995)); and measurement of radioactivity incorporated during amplification (see, e.g., Innis et al., supra). Other suitable methods for detecting amplified products include using dual labeled probes, e.g., probes labeled with both a reporter and a quencher dye, which fluoresce only when bound to their target sequences; and using fluorescence resonance energy transfer (FRET) technology in which probes labeled with either a donor or acceptor label bind within the amplified fragment adjacent to each other, fluorescing only when both probes are bound to their target sequences. Suitable reporters and quenchers include, for example, black hole quencher dyes (BHQ), TAMRA, FAM, CY3, CY5, Fluorescein, HEX, JOE, LightCycler Red, Oregon Green, Rhodamine, Rhodamine Green, Rhodamine Red, ROX, TAMRA, TET, Texas Red, and Molecular Beacons.

The amplification and detection steps can be carried out sequentially, or simultaneously. In a preferred embodiment, RealTime PCR is used to detect target sequences. For example, in a preferred embodiment, Real-time PCR using SYBR® Green I can be used to amplify and detect HCV nucleotides (see, e.g., Ponchel et al., BMC Biotechnol. 3:18 (2003)). Specificity of the detection can conveniently be confirmed using melting curve analysis.

D. Detection of HCV Polypeptides

In addition to the detection of infectious HCV using by detecting HCV nucleotides, infectious HCV can also be detected by detecting HCV polypeptides (e.g., envelope glycoproteins E1 and E2; nonstructural proteins NS1 and NS2, and HCV core antigen) using multiple immunoassays known in the art. For example, HCV polypeptides can be detected using HCV-specific IgG isolated from HCV-infected individuals. Alternately, HCV polypeptides are detected using polyclonal or monoclonal antibodies that specifically bind to HCV polypeptides. For a review of suitable immunological and immunoassay procedures, see, e.g., Harlow & Lane, ANTIBODIES, A LABORATORY MANUAL, Cold Spring Harbor Publication, New York (1988); Basic and Clinical Immunology (Stites & Terr eds., 7^{th} ed. 1991); U.S. Pat. Nos. 4,366,241; 4,376,110; 4,517,288; and 4,837,168); Methods in Cell Biology: Antibodies in Cell Biology, volume 37 (Asai, ed. 1993).

HCV-specific IgG can be isolated from HCV-infected individuals using any means known in the art. Typically, total proteins are precipitated from serum from an HCV-infected individual patient and solubilized. Insoluble particles are removed by centrifugation. The IgG fraction can then be isolated from the solubilized serum proteins using, e.g., a Protein A or Protein G column. The isolated IgG can be further purified by flow filtration and used directly in an assay to detect HCV polypeptides or stored until use.

Methods of producing polyclonal and monoclonal antibodies that react specifically with HCV antigens are known to those of skill in the art. For example, preparation of polyclonal and monoclonal antibodies by immunizing mice with an appropriate immunogen (e.g., a naturally occurring or recombinant HCV polypeptide) is described in, e.g., Coligan, Current Protocols in Immunology (1991); Harlow & Lane, supra; Goding, Monoclonal Antibodies: Principles and Practice (2d ed. 1986); and Kohler & Milstein, Nature 256:495497 (1975). Antibody preparation by selection of antibodies from libraries of nucleic acids encoding recombinant antibodies packaged in phage or similar vectors is described in, e.g., Huse et al., Science 246:1275-1281 (1989) and Ward et al., Nature 341:544-546 (1989). In addition, antibodies can be produced recombinantly using methods known in the art and described in, e.g., Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

A number of HCV polypeptide immunogens may be used to produce antibodies specifically reactive with HCV polypeptide. Recombinant protein can be expressed in eukaryotic or prokaryotic cells as described above, and purified using methods known in the art. Recombinant protein is the preferred immunogen for the production of monoclonal or polyclonal antibodies. Alternatively, a synthetic peptide derived from the sequences disclosed herein and conjugated to a carrier protein can be used an immunogen. Naturally occurring protein may also be used either in pure or impure form. The product is then injected into an animal capable of producing antibodies. Either monoclonal or polyclonal antibodies may be generated, for subsequent use in immunoassays to measure the protein.

Methods of production of polyclonal antibodies are known to those of skill in the art. An inbred strain of mice (e.g., BALB/C mice) or rabbits is immunized with the protein using a standard adjuvant, such as Freund's adjuvant, and a standard immunization protocol. The animal's immune response to the immunogen preparation is monitored by taking test bleeds and determining the titer of reactivity to the beta subunits. When appropriately high titers of antibody to the immunogen are obtained, blood is collected from the animal and antisera are prepared. Further fractionation of the antisera to enrich for antibodies reactive to the protein can be done if desired (see, Harlow & Lane, supra).

Monoclonal antibodies may be obtained by various techniques familiar to those skilled in the art. Briefly, spleen cells from an animal immunized with a desired antigen are immortalized, commonly by fusion with a myeloma cell (see, Kohler & Milstein, Eur. J. Immunol. 6:511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse, et al., Science 246:1275-1281 (1989).

Monoclonal antibodies and polyclonal sera are collected and titered against the immunogen protein in an immunoassay, for example, a solid phase immunoassay with the immunogen immobilized on a solid support. Typically, polyclonal antisera with a titer of 10⁴ or greater are selected and tested for their cross reactivity against non-HCV proteins, using a competitive binding immunoassay. Specific polyclonal antisera and monoclonal antibodies will usually bind with a Kd of at least about 0.1 mM, more usually at least about 1 μM, preferably at least about 0.1 μM or better, and most preferably, 0.01 μM or better. Antibodies specific only for a particular HCV isotype, such as the HCV 1a can also be made.

Once the specific antibodies against a HCV polypeptide are available, HCV polypeptides can be detected and/or quantified by a variety of immunoassay methods (see, e.g., U.S. Pat. Nos. 4,366,241; 4,376,110; 4,517,288; and 4,837,168). For a review of the general immunoassays, see also Methods in Cell Biology: Antibodies in Cell Biology, volume 37 (Asai, ed. 1993); Basic and Clinical Immunology (Stites & Terr, eds., 7th ed. 1991). Immunological binding assays (or immunoassays) typically use capture reagent (i.e., an antibody) that specifically binds to and immobilizes the analyte (i.e., a HCV polypeptide).

Immunoassays also often use a labeling agent to specifically bind to and label the complex formed by the antibody and antigen. The labeling agent may itself be one of the moieties comprising the antibody/antigen complex. Thus, the labeling agent may be a labeled HCV polypeptide or a labeled antibody that specifically binds to a HCV protein. Alternatively, the labeling agent may be a third moiety, such a secondary antibody, which specifically binds to the antibody/HCV protein (a secondary antibody is typically specific to antibodies of the species from which the first antibody is derived). Other proteins capable of specifically binding immunoglobulin constant regions, such as protein A or protein G may also be used as the label agent. These proteins exhibit a strong non-immunogenic reactivity with immunoglobulin constant regions from a variety of species (see, e.g., Kronval et al., J. Immunol. 111:1401-1406 (1973); Akerstrom et al., J. Immunol. 135:2589-2542 (1985)). The labeling agent can be modified with a detectable moiety, such as biotin, to which another molecule can specifically bind, such as streptavidin. The streptavidin may be bound to a label or detectable group as discussed below. A variety of detectable moieties are well known to those skilled in the art.

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent dyes (e.g., fluorescein isothiocyanate, Texas red, rhodamine, and the like), radiolabels (e.g., ³H, ²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecule (e.g., streptavidin), which is either inherently detectable or covalently bound to a signal system, such as a detectable enzyme, a fluorescent compound, or a chemiluminescent compound. The ligands and their targets can be used in any suitable combination with antibodies that recognize HCV, or secondary antibodies that recognize anti-HCV antibodies.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see, U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally, simple calorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

In preferred embodiments, Western blot (immunoblot) analysis is used to detect the presence of HCV polypeptides. Any format can be used for the Western blot analysis including a dot blot or analysis of HCV proteins separated by gel electrophoresis. For the dot-blot assay, various protein dilutions are dot blotted onto a solid support, typically a nitrocellulose membrane (0.22μ, Micron Separations Inc. Westboro, Mass.). For analysis of HCV proteins separated by gel electrophoresis, proteins are typically separated by SDS-PAGE under non-reducing conditions and transferred to a solid support, typically nitrocellulose membranes. In both cases, the membranes are blocked to minimize nonspecific binding (e.g., with were blocked with nonfat powdered milk, bovine serum albuimun, or gelatin). After blocking, the membranes are contacted with purified HCV-specific antibodies and antibody binding is detected with a labeled secondary antibody.

V. Methods of Screening for Inhibitors of HCV

One embodiment of the invention provides methods of screening to identify compounds that inhibit HCV production. One or more of the HCV-infected cells (i.e., a culture of HCV-infected cells) described herein is contacted with a candidate compound (i.e., a compound suspected of having the ability to inhibit HCV production). The effect of the compound on HCV production is determine by detecting the level of HCV-production by the cell, e.g., by detecting HCV nucleotides or polypeptides using the methods described herein. A compound that decreases the level of HCV production in an HCV-infected cell or cell culture relative to the levels of HCV production in an HCV-infected cell or cell culture that has not been contacted with the compound is identified as an inhibitor of HCV production. Typically an inhibitor of HCV production decreases the level of HCV production by about 10%, 20%, 30%, 40%, 50%, 60%, 70%, 80%, 90% or more relative to the level of HCV production in the absence of the compound.

Suitable candidate compounds include, for example, proteins such as interferons, peptides, agents that induce interferons, agonists of toll like receptor 9, protease inhibitors, nucleic acids such as CpG-containing oligonucleotides, anti-sense oligonucleotides, siRNA, ribozymes, antibodies, and small organic molecules.

In some embodiments, variants of a chemical compound (i.e., a “lead compound”) that inhibits HCV production are created and evaluated for their ability to inhibit HCV production. Often, high throughput screening (HTS) methods are employed for such an analysis.

In one preferred embodiment, high throughput screening methods involve providing a library containing a large number of potential therapeutic compounds (candidate compounds). Such “combinatorial chemical libraries” are then screened in one or more assays to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis by combining a number of chemical “building blocks” such as reagents. For example, a linear combinatorial chemical library, such as a polypeptide (e.g., mutein) library, is formed by combining a set of chemical building blocks called amino acids in every possible way for a given compound length (i.e., the number of amino acids in a polypeptide compound). Millions of chemical compounds can be synthesized through such combinatorial mixing of chemical building blocks (Gallop et al., J. Med. Chem. 37(9):1233-1251 (1994)).

Preparation and screening of combinatorial chemical libraries is well known to those of skill in the art. Such combinatorial chemical libraries include, but are not limited to, peptide libraries (see, e.g., U.S. Pat. No. 5,010,175, Furka, Pept. Prot. Res. 37:487-493 (1991), Houghton et al., Nature, 354:84-88 (1991)), peptoids (PCT Publication No WO 91/19735), encoded peptides (PCT Publication WO 93/20242), random bio-oligomers (PCT Publication WO 92/00091), benzodiazepines (U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with a Beta-D-Glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho, et al., Science 261:1303 (1993)), and/or peptidyl phosphonates (Campbell et al., J. Org. Chem. 59:658 (1994)). See, generally, Gordon et al., J. Med. Chem. 37:1385 (1994), carbohydrate libraries (see, e.g., Liang et al., Science 274:1520-1522 (1996), and U.S. Pat. No. 5,593,853), and small organic molecule libraries (see, e.g., benzodiazepines, Baum, C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; and benzodiazepines, U.S. Pat. No. 5,288,514.

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.), which mimic the manual synthetic operations performed by a chemist. The above devices, with appropriate modification, are suitable for use with the present invention. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

The assays to identify compounds that inhibit HCV production are amenable to high throughput screening. High throughput assays for evaluating the presence, absence, quantification, or other properties of particular nucleic acids or protein products are well known to those of skill in the art. Similarly, binding assays and reporter gene assays are similarly well known. Thus, e.g., U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate procedures, including sample and reagent pipeting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems.

VI. Kits

The present invention also provides kits for detecting HCV, e.g. for diagnostic and/or therapeutic purposes. The kits can be used to detect HCV in a biological sample from an individual suspected of being infected with HCV. The kits can also used to be identify the particular strain of HCV infecting an individual. Kits for detecting HCV nucleotides typically comprise two or more components necessary for amplifying and detecting HCV. Kits for detecting HCV polypeptides typically comprise two or more components necessary to specifically bind HCV polypeptides. Components may be compounds, reagents, containers and/or equipment. For example, one container within a kit may contain a HCV sequence (e.g., a SEQ ID NO: 1) and another container within a kit may contain a set of primers, e.g., SEQ ID NOS: 2 and 3 and 4 and 5; and/or SEQ ID NOS: 6 and 7 and 8 and 9. Alternately, one container within a kit may contain a HCV polypeptide and another container within a kit may contain an antibody that specifically binds to the HCV polypeptide. In addition, the kits comprise instructions for use, i.e., instructions for using the primers and probes in amplification and/or detection reactions as described herein.

The kits may further comprise any of the extraction, amplification, detection reaction components or buffers described herein.

### EXAMPLES

The following examples are provided to illustrate, but not to limit the claimed invention.

### Example 1

### Materials and Methods

Infection of cultured cells with high titer HCV sera. HCV infected patient serum was filtered through 0.45 μl filters (Fisher Scientific) and frozen in 1 ml aliquots at −70° C. A fresh vial of frozen serum was used for every new transmission experiment. The cells were infected using 500 μl of thawed donor serum (see, Salahuddin et al., Science 234:596-601 (1986) and Salahuddin et al., Science 234:596-601 (1986)).

Generation of macrophages. Macrophages were generated from human cord blood mononuclear cells (CBMCs) by treating with Phorbol-12-myristate-13-acetate (PMA, 5 ng/ml in complete medium) (Salahuddin et al., Science 242:430-433 (1988)). A majority of the cells that adhered to the plastic were positive for non-specific esterase and phagocytosis, which are established markers for all macrophages. Multiple flasks (Falcon 3108 and 3109) were prepared in all cases to be used separately either for infection with HCV sera or for coculture with the infected patient's PBMC. The non-adherent cells contained approximately 60% CD19 and CD20 positive B-cells, with T-cells and monocytes accounting for the remainder. The cells that did not stain for macrophage-specific markers or phagocytosis were designated as non-committed lymphoid cells, and then infected either with HCV using 500 μl sera or cocultured with PBMC from the same patient.

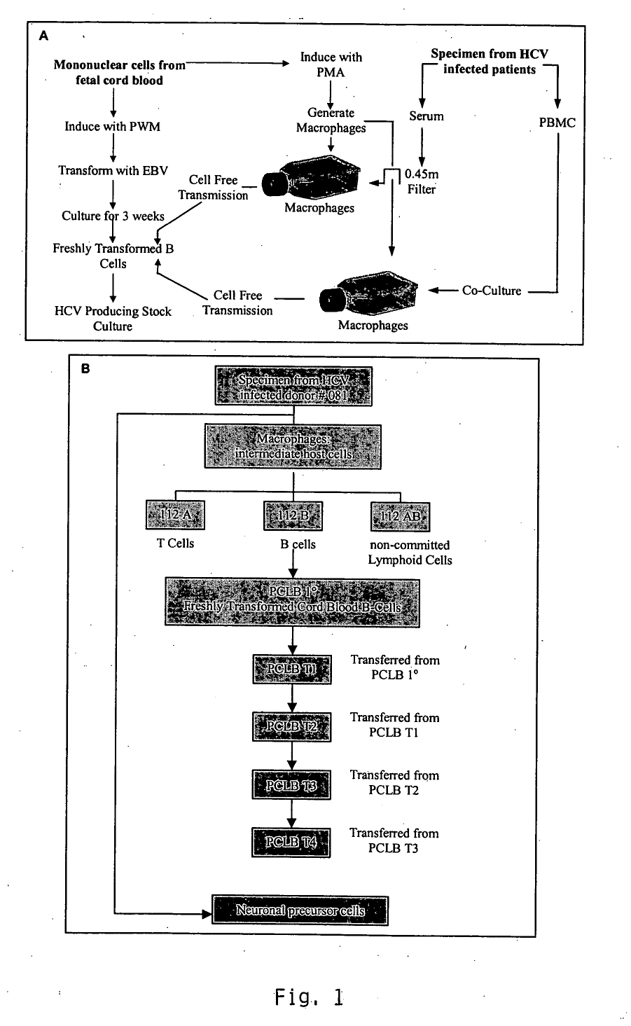

Infection of macrophages with HCV. The macrophages were first treated overnight with polybrene (5 ng/ml) and then infected either with 500 μl of sera or cocultured with the PBMC from the same patient (FIG. 1A). These infected macrophages were incubated overnight at 37° C. in a 5% CO₂ atmosphere. Media were changed and the cultures were continued for another six days with changes of media on day four.

Generation of immortalized B-cells. To create immortalized B-cells, CBMCs were stimulated with pokeweed mitogen (PWM, 5 μg/ml in complete culture medium), and then infected with transforming Epstein-Barr virus (EBV). These immortalized B-cells did not produce EBV (see, Fingeroth et al., PNAS USA 81:4510-4514 (1984) and Lusso et al., Human Herpesvirus-6 Epidemiology, Molecular Biology and Clinical Pathology. Vol. 4. Amsterdam, The Netherlands: Elsevier; p. 121-133 (1992)).

Cell free transmission of HCV. Cell culture supernatants from infected macrophages (as described above) were harvested. Harvested supernatants were then filtered through a 0.22μ filter. The target cells were pretreated overnight with polybrene (5 ng/ml). A 500 μl aliquot of the filtered supernatant was used for infecting each of the target cells.

Design of positive- and negative-strand primers. In order to identify HCV-RNA, nested primers for each strand from the 5′ untranslated region (UTR) were designed. The primer sequences are set forth in Table 1.

Detection of positive- and negative-strand HCV-RNA by RT-PCR assay. Total RNA was extracted from infected cell culture supernatants harvested 5 days after a change of media (TRI REAGENT LS, Molecular Research Center Inc. Cincinnati, Ohio). A 278 base pair region was amplified by nested PCR from the highly conserved 5′-UTR of the HCV genome. For the positive strand assay, a 10 μl aliquot of the total extracted RNA was reverse transcribed using the primer HCV 9.2 with the MMLV Reverse Transcriptase (Promega Corp. Madison, Wis.) or with the Sensiscript Reverse Transcriptase (Qiagen Inc. Valencia, Calif.) according to the manufacturers' instructions. A 5 μl aliquot of the cDNA was then amplified by nested PCR using HCV 9.1 and HCV 9.2 as the outside primers, followed by amplification of 5 μl of the first PCR product using HCV 10.1 and HCV 10.2 as the inner primers.

For the negative strand assay, total RNA was extracted from the cells using the Oligotex Direct mRNA purification kit (Promega Corp. Madison, Wis.). A 10 μl aliquot of the total extracted RNA was reverse transcribed using the HCV1 primer with the RTth polymerase (Invitrogen) according to manufacturer's instructions. Nested PCR amplification was then carried out on a 5 μl aliquot of the cDNA using HCV1 and HCV2 as the outer primers, followed by amplification of 5 μl of the first PCR product using the HCV3 and HCV4 as the nested primers under standard PCR conditions. For each PCR, forty cycles of amplification were performed with the following temperature profiles: 94° C. for 1 min, 55° C. for 1 min, and 72° C. for 1 min for the outer primer set and 94° C. for 1 min, 60° C. for 1 min, and 72° C. for 1 min for the inner primer set.

Detection of positive-strand HCV-RNA by Real-time RT-PCR. The total extracted RNA was solubilized in 10 μl of RNase-free water and then reverse transcribed using the primer HCV 10.2 with the MMLV Reverse Transcriptase. A 5 μl aliquot of the cDNA was then amplified by real-time PCR, using HCV 10.1 and HCV 10.2 primers on the Rotor-Gene 200 amplification system (Corbett Research, Australia) and the SYBR Green I fluorescent dye (BioWhittaker Molecular Applications, Rockland, Me.), using the manufacturers' instructions. An in vitro transcribed RNA from the HCV 5′-UTR was utilized as the standard. Forty cycles of amplification were performed with the following temperature profile: 94° C. for 1 min, 55° C. for 1 min, and 72° C. for 1 min.

Detection of HCV-RNA by in situ hybridization. Approximately 6×10⁴ cells were centrifuged (Cytospin II, Shandon, Pittsburgh, Pa.) onto RNase-free Poly-L-lysine coated slides (Fisher Scientific, Pittsburgh, Pa.), forming a uniform well spread mono layer of cells. These cells were fixed and desiccated with ethanol. Cells were then rehydrated with 1×SSC buffer and treated for protein digestion with proteinase K (Fisher) and for permeation with retentive (ICN Biomedicals, Aurora, Ohio). Hybridization of the probes to the cells was performed overnight at 56° C. After overnight hybridization, to minimize the amount of unhybridized probes, cells were washed three times with formamide followed by one wash with RNAse A and one wash with RNAse-free RNase buffer. Depending upon the batch of reagents, the slides were coated with liquid emulsion (K5 Liquid Emulsion, Ilford Imaging, UK) and exposed for 10-15 days. After exposure, the slides were developed with Kodak D19 developer (Eastman Kodak Company, Rochester, N.Y.) and fixed using the Ilford Hypam Fixer (Ilford Imaging, UK). The developed slides were then stained with Wright-Gimsa Stain (EM Diagnostics Systems, Gibbstown N.J.) and mounted with permount. The probes, used for in situ hybridizations, were prepared by cloning a DNA sequence corresponding to the 5′ untranslated region (5′-UTR), nucleotides 55-308, of HCV RNA into pGEM-T Easy vector (PROMEGA Corp. Madison, Wis.). S³⁵-labeled probes, complementary to the positive- or negative-strand of HCV-RNA, were generated by in vitro transcription in the presence of a ³⁵S rUTP (Amersham Biosciences, England) using the appropriate RNA polymerases as supplied by the manufacturer (Promega Corp. Madison, Wis.) and purified through Sephadex G50 (38).

The sequence of the probe obtained from automated DNA sequencing is as follows: gcactcgcaagcaccctatcaggcagtaccacaaggcctttcgcgacccaacactactcggctagcagtctcgcgggggcacgccc aaatctccaggcattgagcgggttgatccaagaaaggacccggtcgtcctggcaattccggtgtactcaccggttccgcagaccacta tggctctcccgggagggggggtcctggaggctgcacgacactcatactaacgccatggctagacgctttctgcgtgaagacagtagtt cctcacagg.

Genotyping of CIMM-HCV. RNA was extracted and amplified via RT-PCR using the positive-strand RT-PCR assay primer set as described before. Products of the RT-PCR were cloned into the PCR 4.1 cloning vector (Invitrogen Corp. Carlsbad, Calif.). Plasmid DNA was isolated from individual clones and sequenced on an ABI 377 automated DNA sequencer using a Dye Terminator Sequencing Kit (Applied Biosystems, Foster City, Calif.).

Purification of Immunoglobulin (IgG) from HCV infected sera. The IgG fraction was eluted from patient #081 sera using an Affi-Gel II Protein A column (Bio-Rad Laboratories, Hercules, Calif.). Purified IgG were concentrated by Microcon 50 columns (Millipore Corp., Billerica, Mass.) and stored at −20° C.

Extraction of viral proteins from cell culture supernatants. Total proteins were precipitated from the cell culture supernatant or patient serum with the TR1 REAGENT (Molecular Research Center, Inc. Cincinnati, Ohio). The ethanol washed protein pellet was solubilized into 200-500 μl of 1% SDS by incubating at 55° C. for 10 minutes. Any remaining insoluble subcellular particles were removed by centrifugation at 14000×g for 10 minutes at 4° C. Proteins were quantified using the Bradford Protein Assay (Sigma-Aldrich Corp. St. Louis, Mo.) and frozen (−20° C.).

Dot-blot assay and Western analysis. For the dot-blot assay, various protein dilutions (undiluted to 10⁻³) were dot blotted onto a nitrocellulose membrane (0.22μ, Micron Separations Inc. Westboro, Mass.). For the Western analysis, proteins were separated by SDS-PAGE under non-reducing conditions and transferred to nitrocellulose membranes (Bio-Rad Labs). The membranes were blocked with 2% non-fat milk in 20 mM TBS, 500 mM NaCl, 0.02% Tween for 1 hour. The samples were then incubated with purified IgG (1:1000 dilution) for 2-4 hours at room temperature. Antibody binding was detected by incubation with alkaline phosphatase-conjugated goat anti-human antibodies followed by color development (Bio-Rad) (see, Josephs et al., Science 234:601-603 (1986)).

### Example 2

### Identification of Macrophages as Suitable Intermediate Host Cells for Infectious HCV Production

Our initial experiments to develop an in vitro system of HCV replication were performed as previously reported by many investigators using a large variety of established cell lines comprising of various cell types (see, Kato and Shimotohno, Curr Top Microbiol Immunol. 242:261-278 (2000)). These included human transformed liver cells in addition to Hela, CEM, H9, Jurkat, Molt 3, Molt 4, U937, P3HR1, Raji, Daudi, human foreskin fibroblast (ATCC, Bethesda, Md.). All of these cell types could be infected by the reported methods, with the exception of human foreskin fibroblasts, which was uninfectable (Table 2). Results from these efforts did not prove to be reproducible for both isolation and sustained replication of HCV. We were, however, able to detect negative-strand (replicative) RNA for HCV in a few B-cells, liver cells, and macrophages. However, none of the standard cell lines produced infectious HCV for further transmission. These cultures eventually became negative for HCV-RNA, leaving the uninfected cells to grow. Cell line U937, despite its monocytic origin and detectable positive- and negative-strand HCV-RNA, had very low levels of viral RNA expression.

Because our initial experiments provided no significant improvement over the previously reported findings, we used a different approach for HCV isolation. We noted that macrophages were important cells for the transmission of HCV due to their higher incidence of infection and RNA levels. This was analogous to the infection of similar cells with human immunodeficiency virus (HIV-1) (see Moriuchi et al., PNAS USA. 93:15341-15345 (1996)). Therefore, we initiated the use of freshly isolated cells in our laboratories in place of the established cell lines. We tested endothelial cells from fresh fetal umbilical cord, mononuclear cells from fetal cord blood, macrophages from cord blood mononuclear cells (CBMC), peripheral blood mononuclear cells (PBMC), and Kupffer's cells and hepatocytes from fresh liver biopsies. These freshly obtained cells were infectable with HCV and expressed both the positive- and the negative-strands of HCV-RNA.

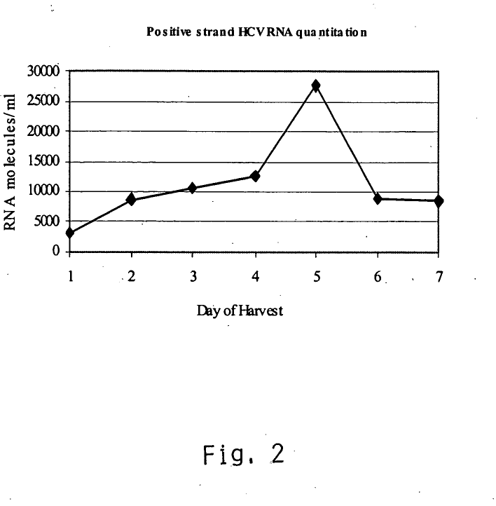

Further experiments were designed that used macrophages as the intermediate host. The results from macrophage cultures were most encouraging. Therefore, we decided to combine the macrophages with B-cells into one system. HCV behaved as a lytic virus for the infected B-cells, with cell death increasing from ˜5% in controls to 20% in infected cultured cells. These infected B-cells formed enlarged cells which eventually died without further replication (FIG. 2C). Retransmissions were achieved by using the culture supernatants obtained from macrophages that had been freshly prepared and infected in our laboratories. It also became apparent that in order to carry the transmitted virus for an extended period of time in vitro, long-lived B-cells were required. We opted in favor of freshly immortalized B-cells.

### Example 3

### Long Term Production of Infectious HCV

To show that our system could be used to grow HCV for extended periods, we tested each isolate at regular intervals by RT-PCR and retransmission into fresh cells (Table 3). Due to the large number of samples that were tested, HCV isolation and long term replication were carried out in several phases: short term cultures (positive for HCV up to 10 weeks), medium term cultures (positive for 10-23 weeks), or extended term cultures (positive for over 23 weeks). An example of a long term positive cell culture is isolate #081. This isolate was obtained from similarly numbered serum from donor #081. Isolate #081 has been maintained in culture for over one hundred thirty weeks. This was designated as the index isolate: CIMM-HCV. Isolate #081 has been propagated in different cell types such as enriched B-cells, T-cells, and non-committed lymphoid cells by both co-culture and cell-free methods. Serial transmissions to freshly transformed B-cells were performed by cell-free methods for further analysis (FIG. 1B). Cell culture supernatants were harvested at least every month and assayed for positive-strand HCV-RNA by nested RT-PCR analysis (Table 3). Due to the consistently positive assays over a period of many months, the isolated HCV was considered to be infectious and replicating virus in new cells.

### Example 4

### Host Range of Infectious HCV Isolates

CIMM-HCV is maintained in one cell type: freshly transformed B-cells, since they are very long-lived as compared to any other cell. In order to establish the host range of this isolate, a large number of cell types were tested for HCV propagation as described before. In addition to B-cells and macrophages, neuronal precursors were found to be injectable as well. These neuronal cells became a significant producer of infectious HCV (Table 4). The neuronal cells survived better after HCV infection in terms of cell viability in comparison to B cells (FIGS. 2A and 2B). Cell-free CIMM-HCV was transmitted to our two neuronal cell types, T (telencephalon) and M (metencephalon), which subsequently showed replication of transmissible infectious virus (experiment 244). Virus from these cells was transmitted to fresh T and M neuronal cell cultures in experiment 248 and from 248 to 260 (Table 4). Infections of neuronal cells were repeated several times with similar results with respect to HCV production and cell lysis. We have since transmitted this HCV from experiments 260 to 273 and 273 to 277.

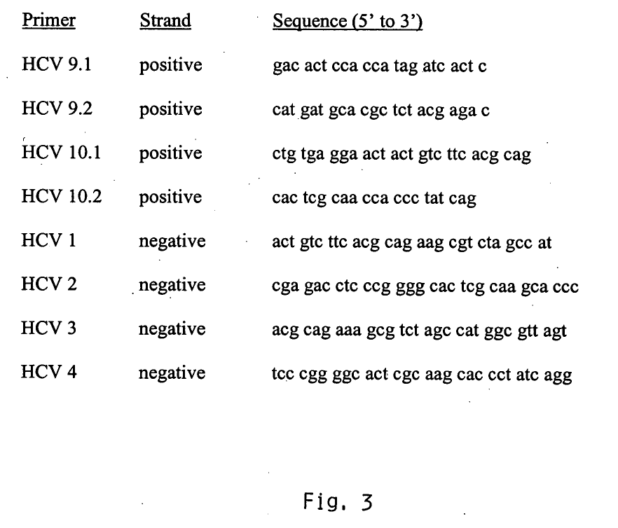

The results of the nested RT-PCR assays for the positive- and negative-strands of CIMM-HCV RNA from different cell cultures are shown in FIG. 3C. The presence of the expected 278 base pair PCR product demonstrated that positive- and negative-strands of HCV-RNA were present in our system, indicating both replication and extracellular production of the virus.

### Example 5

### Conformation of Infectious HCV Isolation

We obtained 151 peripheral blood specimens from HCV infected patients and 5 uninfected controls who volunteered to donate their blood. All specimens were acquired with the approval of the Institutional Review Board (IRB) and donors' informed consent. Specimens were obtained from 111 Caucasians, 39 Hispanics and 6 African Americans. The participants included 108 males and 48 females. All specimens were freshly processed within an hour of blood drawing. Repeat samples were obtained from 77 of the original patients in order to confirm our initial results. Thirty-three of these 151 patients were co-infected with HIV-1, and the remainder of the donors had hematological malignancies or other cancers. Using our system, HCV was isolated with 75% efficiency from these 151 specimens. No HCV was ever isolated from the 5 uninfected controls. This high rate of isolation of HCV shows that this system is useful in obtaining HCV from a variety of individual patients for further analysis.

### Example 6

### Determination of Optimum Day for Harvesting Infectious HCV for RNA Extraction

In order to determine the optimum day for harvesting the highest accumulation of positive-strand RNA, a CIMM-HCV culture was divided into 7 separate flasks, each containing approximately 10⁶ cells. On day zero, fresh media was added to each flask. For each of the next seven days, one flask was harvested and assayed for the positive- and negative-strands of HCV-RNA using nested PCR. RNA in the media was assayed for the positive strand, while the whole cell RNA was used for assaying for the negative strand. While day 5 showed the greatest accumulation of positive-strand of HCV-RNA, the levels of the negative-strand on all seven days remained unchanged.

Changes in the overall levels of HCV-RNA should reflect the sum of the RNA production and RNA destruction. This observed periodicity in the positive-strand therefore, may be due to: (1) slowing of the replication process of the infected cells or from in situ production of an inhibitor; or (2) lysis of infected cells causing destruction of the virus and its RNA, e.g. by released proteases and ribonucleases. Stability of the negative-strand inside the cells was not a surprise, as the RNA used for the assay was obtained by lysing chronically infected cells.

In an experiment performed simultaneously, the positive-strand HCV-RNA in the cell culture supernatants was analyzed quantitatively by real-time RT-PCR. Approximately 3200 copies of HCV-RNA at day zero increased during the experiment to ˜27,000 copies per ml on day 5 and then progressively decreased (FIG. 3). This data confirmed the pattern obtained using the nested RT-PCR assay.

### Example 7

### Detection of HCV-RNA by In Situ Hybridization

We analyzed our HCV infected cells by performing in situ hybridizations to visualize the percentage of infected cells and the locations of the HCV-specific strands (see, Moldvay et al., Blood 83:269-273 (1994)). The uninfected cells used as a control did not hybridize to either negative or positive strand probes. In all cases, the background grains were light. Hybridization with the probe for the positive-strand produced a halo-like appearance around the periphery of the infected cells. A strong signal for the negative strands of HCV-RNA was seen confined within the cells, possibly in the cytoplasm. Although approximately 5% of the cells appeared strongly positive, this may have been an underestimate due to: (1) cell lysis of infected cells in culture; and (2) the loss of cells that attach to the filter cards used in preparing the cytospin slides. Hybridization to both the positive- and negative-strands of HCV-RNA suggests replication and production of HCV. Results of the in situ hybridizations are consistent with the nested RT-PCR assay. A majority of the infected cells appear to be large; however, there were a significant number of smaller cells that also gave lighter positive signals. By comparison, neither the enlarged cells nor the small ones in the control population showed any positive signal. We believe that the small, infected cells probably progressively enlarge, produce virus, and die. This phenomenon is also observed in human immunodeficiency virus (HIV) and HHV-6 infected cell cultures (see, Lusso, Human Herpesvirus-6 Epidemiology, Molecular Biology and Clinical Pathology. Vol. 4. Amsterdam, The Netherlands: Elsevier; p. 25-36 (1992)).

### Example 8

### Genotyping of the HCV Isolate

Based on sequence analysis, HCV has been classified into six major genotypes and a series of subtypes (see, Simmonds et al., J Gen Virol. 74:2391-2399 (1993)). The highly conserved 5′ untranslated region (5′-UTR), routinely used for RT-PCR detection of HCV-RNA, exhibits considerable genetic heterogeneity (see, Bukh et al., PNAS USA. 89:4942-4946 (1992)) and shows specific polymorphism between types and subtypes. This genetic heterogeneity of the 5′-UTR has been utilized for the genotyping of HCV (see, Chan et al., J Gen Virol. 73:1131-1141 (1992); Davidson et al., J Gen Virol. 76:1197-1204 (1995); Krekulova et al., J Clin Microbiol. 39:1774-1780 (2001); O'Brien et al., Dig Dis Sci. 42:1087-1093 (1997); Stuyver et al., J Gen Virol. 74:1093-1102 (1993); and White et al., J Clin Microbiol. 38:477-482 (2000)). In order to identify the genotype of CIMM-HCV, we cloned and sequenced the 5′-UTR. Based on the sequence homology searches, CIMM-HCV was similar to genotype 1a.

In order to spot check the genome of CIMM-HCV, we tested most of the previously published primers (see, e.g., Chayama, Hepatitis C Protocols, J. Y. Lau Ed., vol 19 of Methods in Molecular Medicine (Humana Press, Totowa, N.J., 1998), pp. 165-173; Koylkhalov, et al., Hepatitis C Protocols, J. Y. Lau Ed., vol 19 of Methods in Molecular Medicine (Humana Press, Totowa, N.J., 1998), pp. 289-301; Norder et al., J. Clin. Micro. 36, 3066-3069 (1998); and Rispeter et al., J. Gen. Virol. 78, 2751-2759 (1997)). Primers described in Chayama, are labeled as (1); primers described in Koylkhalov, et al., are labeled as 2; primers described in Norder et al., are labeled as (3); primers described in Rispeter et al., are labeled as (4); primers that we developed are labeled as (5). We, however, found that many of these primers did not lead to RT-PCR products from our isolate (Table 5). We attribute this to the heterogeneity of HCV RNA (Bukh et al., PNAS USA. 89:187-191 (1992)). It is possible that parts of our isolate may differ significantly from the previously reported sequences. We are currently in the process of sequencing the entire CIMM-HCV genome. Although the culture system described here is capable of isolating HCV from approximately 75% of infected patients, the process may select a specific genotype, which may be a more competent and infectious strain, e.g. type 1a or a variation thereof.

### Example 9

### Reactivity of IgG from HCV-Infected Patients

To determine if major HCV proteins are present in the sera of infected patients, polyclonal IgG was purified from patient serum. To determine the reactivity of the freshly eluted polyclonal IgG, various dilutions of the total protein preparations from cell culture supernatants were analyzed. A positive reaction was noted with homologous serum proteins using CIMM-HCV obtained from B-cell supernatant, supernatants from neuronal cells (from transmission experiment 260), and commercially available HCV core antigen (ViroGen Corp. Watertown, Mass.). No reaction was seen with proteins from uninfected cells or with the NS4 antigen (ViroGen Corp.). These results show that IgG purified from patient's sera specifically detects HCV proteins, particularly core antigen.

### Example 10

### Analysis of HCV Proteins

The HCV genome encodes a polyprotein which is subsequently processed into a number of mature structural and nonstructural moieties (see, Grakoui et al., J. Virol. 67:2832-2843 (1993)). The host cell signal peptidases cleave the N-terminal region of the precursor polypeptide to produce the HCV core protein (see, Harada et al., J. Virol. 65:3015-3021 (1991); Hijikata et al., PNAS USA. 88:5547-5551 (1991); and Selby et al., J Gen Virol. 74:1103-1113 (1993)). The HCV core protein is reported to range between 16 and 25 kDa in size, however, it is possible that the size differences that have been previously reported may be due to differences in processing of the HCV core protein (see, Hijikata et al., PNAS USA. 88:5547-5551 (1991); Lo et al., Virology 199:124-131 (1994); Yasui et al., J. Virol. 72:6048-6055 (1998); and Yeh et al., J Gastroenterol Hepatol. 15:182-191 (2000)).

To determine whether the replicating CIMM-HCV was producing major HCV proteins, Western blot analyses using non-reducing conditions were performed. The polyclonal IgG detected a series of proteins in the HCV positive patient sera and in the infected cell culture supernatant. Proteins of 140, 75, 50, 37, 32, 27 and 25 kDa were detected in these samples. The polyclonal IgG also gave a positive reaction with the commercially obtained recombinant core antigen. This core antigen has β-galactosidase fused at the N-terminus and is thus approximately 140 kDa in size, as reported by the manufacturer. We discuss these protein bands below.

There are two highly glycosylated envelope proteins, E1 (32 and 35 kDa) and E2 (70 kDa) (see, Blanchard et al., J. Virol. 76:4073-4079 (2002); Dubuisson et al. J Virol. 68:6147-6160 (1994); Hijikata et al., PNAS USA. 88:5547-5551 (1991); and Lanford et al., Virology 197:225-235 (1993)). A band at approximately ˜140 kDa was seen in all of the infected cell culture supernatants. This and the higher molecular weight bands may have resulted from the multimerization of core, E1 and E2, or homodimerization of E2. The E1 and E2 proteins are known to form non-covalently linked heterodimers under non-reducing conditions (see, Deleersnyder et al., J Virol. 71:697-704 (1997) and Dubuisson et al. J Virol. 68:6147-6160 (1994)). The Core and E1 proteins also bind to each other (see, Lo et al., J Virol. 70:5177-5182 (1996) and Matsumoto et al., Virology 218:43-51 (1996)), and possibly form HCV and host cellular protein complexes as well. An approximately 75 kDa protein was also detected in all of the infected samples that were analyzed. This protein corresponds to the known molecular weight of E2. Proteins in the 32 and 37 kDa range were also detected in the Western blots. These bands are consistent with known sizes of E1.

In all the infected samples, a major protein band of approximately 50 kDa was seen. This was perhaps due to the incomplete processing of the precursor polyprotein (see, Yasui et al., J Virol. 72:6048-6055 (1998)). Since the band was present only when protein purified from infected cell culture supernatants were used for the analyses, the band is therefore related to HCV proteins. Bands of approximately 25 and 27 kDa were also detected. We believe that the core protein, as expressed by wild type HCV in infected cell cultures, may be larger than has been previously described.

### Example 11

### Long Term Production of Infectious HCV In Vitro

It has previously been reported that immortalized B-cells are able to propagate HCV in vitro for varying periods of time (see, Sung et al., J. Virol. 77:2134-2146 (2003)). Although B-cells that are not immortalized can produce HCV, these cell cultures have limited life spans. Freshly prepared macrophages in our system are the intermediate host for HCV isolation. We have not explored the mechanism of infection or replication of HCV, but macrophages from a variety of sources appear to serve this function. It is possible that macrophages modify the HCV sufficiently to enable them to infect other cell types. For example, the E1 and E2 proteins are glycosylated (see, Deleersnyder et al., J Virol. 71:697-704 (1997); Dubuisson et al. J Virol. 68:6147-6160 (1994); and Lanford et al., Virology 197:225-235 (1993)). Alterations in the glycosylation pattern could affect the infectious capability of the progeny virus, and also define the host range. Another possibility is that the macrophages allow accumulation of replicative HCV, along with suitable changes in glycosylation, facilitate a higher level of infection of target cells. In bacteria, infecting phage can have their DNA modified by the host modification system. This allows the phage to escape the restriction system, thus enabling better infection of new hosts. It is therefore not too difficult to imagine that various types of animal cells could produce slightly different versions of infecting viruses, allowing the viruses to preferentially infect different types of tissues. The modifications could be to any part of the virus, but would most likely be to the envelope proteins.

As stated before, we discovered that neuronal precursors were injectable with CIMM-HCV and are significant producers of infectious virus. We have done this repeatedly with a number of other isolates. Neuronal cells are similar to other macrophages both in staining characteristics and in functional assays. However, they are growth factor dependent and are positive for neuronal markers. They have been in culture for over two years. Neuronal T cells grow in large non-adherent and adherent clumps and the M cells are generally adherent and form neuronal cell-like processes. Macrophages from other sources, e.g. Kupffer's cells from liver, get infected, but after the initial few days, gradually lose virus production. HCV-RNA, however, can be detected for several weeks. This may be related to their maturation, cytostasis, and to eventual death. Similar experiments were performed with freshly cultured endothelial cells obtained from human umbilical cord. These cells are related to hepatocytes from liver, both being endothelial cells. The results from these cells were similar to the data from Kupffer's cell analysis.

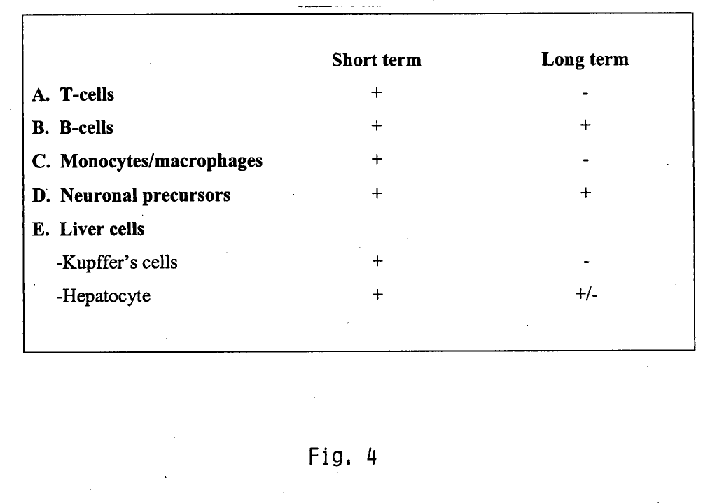

Since this is the first in vitro system for culturing HCV, we have been able to make initial observations regarding replication of the virus. Further studies related to HCV replication and pathogenesis are in progress. The in situ hybridization results seen in FIG. 4 suggest that the positive strand of HCV is synthesized at or migrates to the plasma membrane and that the negative strand remains in the cytoplasm. This observation can only be made in a dynamic system with actively replicating virus. This suggestion is supported by recent reports that RNA-dependent RNA polymerase contains a transmembrane segment which is anchored in the membrane (see, Ivashkina et al., J. Virol. 76:13088-13093 (2002)). Non-structural proteins and positive strand RNA have also been found associated with the plasma membranes (see, Gosert et al., J. Virol. 77:5487-5492 (2003)). These results suggest that the site where HCV is fully assembled is probably in or near the plasma membrane of the infected cells. Probably HCV-RNA is synthesized in the cytoplasm and migrates to the plasma membrane for the final assembly. The completed virion is then released into the extracellular space.

Data from Western blot analysis of the HCV proteins in the cell culture supernatants shows that all of the expected major structural proteins are present as shown by binding to the polyclonal IgG purified from HCV positive patient sera. These specific bindings were not seen in the samples from uninfected cell culture supernatants. Taken together, these results suggest that there is production of HCV specific proteins in CIMM-HCV infected cell cultures.

In addition to the molecular analysis which establishes that our cells are producing HCV virions, the serial transmission of HCV to fresh uninfected cells via cell-free culture supernatants establishes biological evidence of infectious virus (FIG. 1B: PCLB T1-T4). Since this virus is infectious and all of the major proteins appear to be present, the virus that has been grown in culture most likely contains the entire genome.

Our system has allowed us to reproducibly isolate HCV from a majority of patients and in a few cases these cell cultures have been carried for over 130 weeks or over. The amount of HCV produced from this system was sufficient to conduct biological, molecular, and immunological investigations. The analogy between macrophage-initiated in vitro propagation of HIV and HCV is rather remarkable. The dendritic cell-specific ICAM-grabbing non-integrins (DC-SIGN) can bind HIV, and protect it for protracted periods to concentrate and deliver the virions to cause infection of T-cells in trans with high efficiency (12, 31). The structural basis for selective recognition of oligosaccharides on virion envelope proteins by DC-SIGN and DC-SIGR may indeed be a common pattern by which HIV and HCV are concentrated for in vitro transmission to their respective susceptible cells, T cells for HIV and B cells for HCV (see, Feinberg et al., Science 294:2163-2166 (2001) and Pohlmann et al., J Virol. 77:4070-4080 (2003)). Unlike CD4 for HIV, CD81 reported as a possible HCV receptor is currently a subject of serious discussions (see, Masciopinto et al., Virology 304:187-196 (2002); Pileri et al., Science 282:938-941 (1998); and Sasaki et al., J Gastroenterol Hepatol. 18:74-79 (2003)).

Our system allows production of large quantities of HCV. Having a reliable and long-term growth system for HCV in cell culture will facilitate in vitro studies and also aid in the production of rational drugs and vaccines. This culture system will, therefore, allow researchers in the field of HCV and liver disease to perform a wide variety of further analyses that can help in understanding the life cycle of HCV and the mechanisms of pathologies induced in human hosts.

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, and Genbank Accession Nos. cited herein are hereby incorporated by reference in their entirety for all purposes.

1. A method for replicating infectious hepatitis C virus (HCV) in vitro, the method comprising the steps of:
(a) contacting macrophages in vitro with a composition comprising HCV under conditions suitable for infection of the macrophages with HCV,
(b) culturing the infected macrophages in vitro;
(c) obtaining a culture supernatant comprising infectious HCV from the infected macrophages;
(d) contacting non-macrophage cells with the culture supernatant under conditions suitable for infection of the non-macrophage cells with HCV; and
(e) culturing the non macrophage, HCV-infected cells in vitro under conditions suitable for HCV production, thereby replicating infectious HCV in vitro.

2. The method of claim 1, wherein the macrophage and the non-macrophage cells are human.

3. The method of claim 1, wherein the macrophages are primary cells.

4. The method of claim 1, wherein the macrophages are isolated from fetal cord blood.

5. The method of claim 1, wherein the macrophages are obtained by culturing mononuclear cells under conditions suitable for inducing differentiation of the mononuclear cells into macrophages.

6. The method of claim 1, wherein the composition comprising HCV is serum from an HCV-infected subject.

7. The method of claim 1, wherein the composition comprising HCV is peripheral blood mononuclear cells from an HCV-infected subject.

8. The method of claim 1, wherein the non-macrophage cells are primary cells.

9. The method of claim 1, wherein the non-macrophage cells are immortalized.

10. The method of claim 1, wherein the non-macrophage cells are selected from the group consisting of: EBV-immortalized B cells, T cells, non-committed lymphoid cells, and neuronal precursor cells.

11. The method of claim 1 wherein the non-macrophage cells are EBV-immortalized B cells.

12. The method of claim 1 wherein the non-macrophage cells are neuronal precursor cells.

13. The method of claim 12, wherein the neuronal precursor cells are selected from the group consisting of: metencephalon cells and telencephalon cells.

14. The method of claim 1, wherein the HCV-infected cells of step (c) are passaged and produce infectious HCV for at least 23 weeks.

15. A method for isolating infectious hepatitis C virus (HCV) particles from an in vitro culture, the method comprising the steps of:
(a) contacting macrophages with a composition comprising HCV under conditions suitable for infection of the macrophages with HCV
(b) culturing the infected macrophages in vitro;
(c) obtaining culture supernatant comprising infectious HCV from the infected macrophages;
(d) contacting non-macrophage cells with the culture supernatant under conditions suitable for infection of the cells with HCV;
(e) culturing the HCV-infected non-macrophage cells under conditions suitable for HCV production; and
(f) isolating HCV particles from culture supernatant of the HCV-infected non-macrophage cells.

16. The method of claim 15, further comprising the step of:
(g) contacting different non-macrophage cells with the culture supernatant, thereby infecting the different non-macrophage cells with HCV in vitro.

17. The method of claim 16, wherein the macrophage and the non-macrophage cells are human.

18. The method of claim 16, wherein the macrophages are isolated from fetal cord blood.

19. The method of claim 16, wherein the composition comprising HCV is serum from an HCV-infected subject.

20. The method of claim 16, wherein the composition comprising HCV is peripheral blood mononuclear cells from an HCV-infected subject.

21. The method of claim 16, wherein the non-macrophage cells are selected from the group consisting of: EBV-immortalized B cells, T cells, non-committed lymphoid cells, and neuronal precursor cells.

22. The method of claim 16 wherein the non-macrophage cells are EBV-immortalized B cells.

23. A method of screening for compounds that inhibit of HCV production, the method comprising
(a) contacting macrophages in vitro with a composition comprising HCV under conditions suitable for infection of the macrophages with HCV;
(b) culturing the infected macrophages in vitro;
(c) obtaining culture supernatant comprising infectious HCV from the infected macrophages;
(d) contacting non-macrophage cells with the culture supernatant under conditions suitable for infection of the cell with HCV;
(e) contacting the non-macrophage, HCV-infected cells with a compound suspected of having the ability to inhibit HCV production and culturing the HCV-infected cell under conditions suitable for HCV production; and
(f) detecting the level of HCV production in the HCV-infected cell, wherein a compound that decreases the level of HCV production in the HCV-infected cell relative to the level of HCV production in a HCV-infected cell that has not been contacted with the compound, is identified as a compound that inhibits HCV production.

24. The method of claim 23, wherein the compound suspected of having the ability to inhibit HCV production is selected from the group consisting of: an interferon, an agent that induces interferon-α production, a CpG oligonucleotide, an antisense oligonucleotide, an agonist of toll-like receptor 9 (TLR9); and a protease inhibitor.

25. The method of claim 23, wherein the compound suspected of having the ability to inhibit HCV production is a small organic compound.

26. The method of claim 23, wherein the level of HCV production in the HCV-infected cell is detected by detecting the presence of a HCV nucleotide.

27. The method of claim 26, wherein the HCV nucleotide hybridizes under stringent conditions with a oligonucleotide comprising the sequence set forth in SEQ ID NO:1.

28. The method of claim 26, wherein the HCV nucleotide is detected by:
(g) amplifying a HCV nucleotide from a culture supernatant from the HCV-infected cell of step (d) with a pair of oligonucleotide primers comprising the sequences set forth in SEQ ID NOS: 2 and 3 to obtain a first amplified product;
(h) amplifying the first amplified product with a pair of oligonucleotide primers comprising the sequences set forth in SEQ ID NOS: 4 and 5 to obtain a second amplified product; and
(i) detecting the second amplified product.

29. The method of claim 26, wherein the HCV nucleotide is detected by:
(e) amplifying a HCV nucleotide from a culture supernatant from the HCV-infected cell of step (d) with a pair of oligonucleotide primers comprising the sequences set forth in SEQ ID NOS: 6 and 7 to obtain a first amplified product;
(f) amplifying the first amplified product with a pair of oligonucleotide primers comprising the sequences set forth in SEQ ID NOS: 8 and 9 to obtain a second amplified product; and
(g) detecting the second amplified product.

30. The method of claim 23, wherein the level of HCV production in the HCV-infected cell is detected by detecting the presence of a HCV polypeptide.

31. A stable in vitro cell culture for long term replication of infectious HCV, wherein the cells are HCV-infected non-macrophage cells obtained by contacting the non-macrophage cells with a culture supernatant from an in vitro cultured, HCV-infected macrophage, wherein the HCV infected, non-macrophage cells produce infectious HCV.

32. The stable in vitro cell culture of claim 31, wherein at least 80% of the cells produce infectious HCV.

33. The stable in vitro cell culture of claim 31, wherein at least 50% of the cells produce infectious HCV.

34. A stable in vitro cell culture for long term replication of infectious HCV from a single patient isolate, wherein the cells are HCV-infected non-macrophage cells obtained by contacting the non-macrophage cells with a culture supernatant from an in vitro cultured, HCV-infected macrophage, wherein the HCV infected, non-macrophage cells produce infectious HCV.

35. The stable in vitro cell culture of claim 34, wherein at least 80% of the cells produce infectious HCV.

36. The stable in vitro cell culture of claim 34, wherein at least 50% of the cells produce infectious HCV.

37. A stable in vitro cell culture for long term replication of infectious HCV, wherein the cells are HCV-infected non-macrophage cells which produce infectious HCV.

38. The stable in vitro cell culture of claim 37, wherein at least 80% of the cells produce infectious HCV.

39. The stable in vitro cell culture of claim 37, wherein at least 50% of the cells produce infectious HCV.

40. An isolated nucleic acid comprising the sequence set forth in any one of SEQ ID NOS. 1-9.


SEQUENCE LISTING (9 sequences)

SEQ ID NO: 1 - DNA, 271 bp, Hepatitis C virus
  cloned index isolate #081 CIMM-HCV 5′ untranslated region (5′-UTR), probe obtained from automated DNA sequencing
gcactcgcaagcaccctatcaggcagtaccacaaggcctttcgcgacccaacactactcggctagcagtctcgcgggggcacgcccaaatctccaggcattgagcgggttgatccaagaaaggacccggtcgtcctggcaattccggtgtactcaccggttccgcagaccactatggctctcccgggagggggggtcctggaggctgcacgacactcatactaacgccatggctagacgctttctgcgtgaagacagtagttcctcacagg

SEQ ID NO: 2 - DNA, 22 bp, Artificial Sequence
  Description of Artificial SequenceHCV 9.1 positive strand first amplification “outer” primer
gacactccaccatagatcactc

SEQ ID NO: 3 - DNA, 22 bp, Artificial Sequence
  Description of Artificial SequenceHCV 9.2 positive strand first amplification “outer” primer
catgatgcacgctctacgagac

SEQ ID NO: 4 - DNA, 27 bp, Artificial Sequence
  Description of Artificial SequenceHCV 10.1 positive strand second amplification “inner” “nested” primer
ctgtgaggaactactgtcttcacgcag

SEQ ID NO: 5 - DNA, 21 bp, Artificial Sequence
  Description of Artificial SequenceHCV 10.2 positive strand second amplification “inner” “nested” primer
cactcgcaaccaccctatcag

SEQ ID NO: 6 - DNA, 29 bp, Artificial Sequence
  Description of Artificial SequenceHCV 1 negative strand first amplification “outer” primer
actgtcttcacgcagaagcgtctagccat

SEQ ID NO: 7 - DNA, 30 bp, Artificial Sequence
  Description of Artificial SequenceHCV 2 negative strand first amplification “outer” primer
cgagacctcccggggcactcgcaagcaccc

SEQ ID NO: 8 - DNA, 30 bp, Artificial Sequence
  Description of Artificial SequenceHCV 3 negative strand second amplification “inner” “nested” primer
acgcagaaagcgtctagccatggcgttagt

SEQ ID NO: 9 - DNA, 30 bp, Artificial Sequence
  Description of Artificial SequenceHCV 4 negative strand second amplification “inner” “nested” primer
tcccggggcactcgcaagcaccctatcagg